(19) United States
(12) Patent Application Publication
(10) Pub. No.: US 2005/0079491 A1
(43) Pub. Date: Apr. 14, 2005

(54) Method for detecting and identifying the presence of biological substances derived from birds, and oligonucleotides therefor

(76) Inventors: Carole Donne-Gousse, Bois-Guillaume (FR); Vincent Laudet, Lyon (FR); Catherine Hanni, Lyon (FR)

Correspondence Address: YOUNG & THOMPSON, 745 SOUTH 23RD STREET, 2ND FLOOR, ARLINGTON, VA, 22202, US

(21) Appl. No.: 10/275,323

(22) PCT Filed: Apr. 16, 2001
(86) PCT No.: PCT/FR01/01279

(30) Foreign Application Priority Data
May 9, 2000 (FR) 0005850

(51) Int. Cl. C12Q 1/68

(52) U.S. Cl. 435/6

(57) ABSTRACT

The invention concerns a method for detecting the presence of biological substances derived from birds, in particular from anseriformes, galliformes or struthiformes in a sample or organic substance. The invention is characterised in that it consists in determining the presence of DNA of avian origin, in particular derived from goose-type, cock-type or ostrich-type in said organic substance by amplifying a DNA sequence specific of the genome of birds, in particular of anseriformes, galliformes or struthiformes, and contained in the DNA extracted from the sample, namely a sequence present in the genome of birds, in particular anseriformes, galliformes or struthiformes but absent from the genomes of other animal genuses, and in particular of other animal species.

A subject of the present invention is a method of detecting and identifying the presence of biological materials derived from birds, in particular from Anseriformes, Galliformes or Struthioniformes, in a sample of organic material.

Its subject is moreover the oligonucleotides for the carrying out of this method, and the DNA fragments obtained using the said oligonucleotides.

The increasing economic value of certain animal species used to feed humans is linked to a substantial imbalance between supply and demand in the market. A direct consequence of this is the introduction of an increased practice of the adulteration of food.

One of the most common methods of food adulteration comprises the replacement of components derived from animal species with a high commercial value with those derived from species of less value, or even by components of vegetable origin such as soya. Thus for example pig, chicken or even horse meat are frequently used instead of beef, mutton instead of goat meat, or duck instead of goose.

The “inter-specific” adulteration of food products is therefore at one and the same time a problem in terms of economics, public health and preservation of the environment, which affects consumers, distributors, producers as well as the authorities responsible for food hygiene and crime prevention.

It is therefore important to be able not only to determine the presence in food of biological materials derived from animal species, but also to identify the origin of these biological materials present in food.

In the agro-food sector, the characterization of animal species employs biochemical techniques for the analysis of proteins (electrophoresis and immunoanalysis) or the techniques of chemistry (principally chromatography).

Thus, polyacrylamide gel electrophoresis in denaturing conditions was essentially developed for the analysis of cooked food (analysis of peptides or of proteins denatured and coagulated by cooking (Patterson R. L. S., 1985, Biochemical identification of meat species, Elsevier ed)). This technique is at present supplanted by pH gradient electrophoresis (isoelectric focusing or “IEF”), which offers much greater resolution; however, isoelectric focusing is a technique that is tricky to use.

As far as immunoanalysis is concerned, numerous programmes have been devoted to the use of specific antibodies to demonstrate the adulteration of meat products by vegetable proteins, and also to the identification of animal species in dairy products and meats (Hernandez et al., 1994, Food and Agricultural Immunology, 6, 95-104). Antibodies directed against proteins of the muscle sarcoplasm have for example been used to characterize various domestic species such as the pig, the ox, the horse or the chicken. However, immunoanalysis also has major limitations.

As far as analytical chemistry is concerned, it is above all the techniques of liquid- or gas-phase chromatography that have been used to check meat products: determination of peptides in meat (Barai et al., 1992, Trends in Food Science and Technology, 4, 395-401; Carnegie P. R. et al., 1983, Journal of Chromatography, 261: 153-157) or of fatty acids in fats for example (Youssef M. K. E. et al., 1988, Food Chemical, 30: 167-180). However, these techniques lack a real specificity and are limited to the most common species such as the pig.

Although these techniques remain in use for the identification of common species in food (in particular the domestic species), the multiplication in the number of wild species involved (game, fish, crustaceans, shellfish, molluscs) means that the majority of these techniques are at their limits today.

The analysis of the genome by means of nucleic probes obviously represents a new alternative for the identification of species that is still little developed at the present time. In fact, work carried out on old DNA (or fossile DNA) since the start of the 1990s has demonstrated that DNA is a molecule that is very stable after death, despite the action of time and of the environment (Brown et al., 1994, Bioessays, 16 (10): 719-26). However, when it survives in old substrates, this DNA is very degraded and present in small quantities, in the form of damaged and chemically modified molecules (Paabo et al., 1989, Journal of Biology Chemistry. 264, 9709-9712). These characteristics are due essentially to the phenomena of hydrolysis and oxidation (Lindahl, 1993, Nature, 362, 709-715). Thanks to the PCR (“Polymerase Chain Reaction”) technique, which constitutes a tool having a remarkable analytical power, it is possible to multiply a given DNA fragment “in vitro” in a quasi-exponential manner. By amplifying the DNA of a food preparation which has undergone modifications such as cooking, smoking etc., hydrolysis and oxidation phenomena, it will be possible to identify each constituent of animal or vegetable origin. Thus PCR has been recently used for the characterization of cooked pig meat (Meyer et al., 1995, Journal of AOAC International, 78, (6) 1542-1551)), sheep or goat meat (Chikuni et al., 1994, Animal Science and Technology, 65 (6), 571-579) by amplification of sequences specific to the sought species. Similarly, the amplification of sequences specific to the Y chromosome has permitted the determination of the sex of butchery carcasses of bovine and ovine origin (Cotinot C, et al., 1991, Genomics. 10 (3): 646-53, Apparao K. B. C., 1995, Meat Science, 39 (1) 123-126).

A subject of international application WO 98/50401 is a method of detecting the presence of biological materials of bovine origin in a sample of organic material. The method described in this reference employs PCR with the help of suitable oligonucleotides. However, the method described in this reference permits only the detection of the presence or the absence of a single bovine species, namely the species Bos taurus, and thus does not permit the detection and identification of the presence of several bovine species.

Ducks, geese, chickens, turkeys, ostriches are birds that are greatly used in the agro-food sector (foie gras, pätés, meats, fats, cooked dishes, eggs etc.) and are therefore very exposed to adulteration. For example, the foie gras of goose is more expensive and better tasting than that of duck and there is a substantial amount of crime in this area.

Moreover, the adulteration of the aforementioned species can also affect manufactured products (feathers, duvets, costumers with feather accessories etc . . . ), collector's eggs, collector's feathers, bred or wild live animals, stuffed animals etc . . . .

Ducks, geese, chickens, turkeys, ostriches etc . . . belong the Aves class which includes all birds; the Aves form two divisions: the palaeognaths and the neognaths. By way of example, ducks, geese and swans form part of the family of the Anatidae, and belong to the order of the Anseriformes, which forms part of the neognaths. Chickens, quails and turkeys form part of the family of the Gallinaceans and belong to the order of the Galliformes, which also forms part of the neognaths. Ostriches form part of the family of the Struthionidae and belong to the order of the Struthioniformes, which form part of the palaeognaths.

A particular feature of the mitochondrial DNA (mtDNA) of birds is that it is not organized in the same way as that of mammals. In fact, in birds the ND6 gene which codes for component b of the respiratory chain is situated between the replication control region and the gene coding for cytochrome b, whereas in mammals the linkage is as follows: replication control region, gene coding for cytochrome b and ND6 gene (Desjardin et al., 1990, Current Genetics, 17, 515-518).

mtDNA is an excellent marker of species which is often used in phylogeny. In fact, according to the species that is being studied, certain portions of the MtDNA permit differentiation of the species among themselves, others have a finer power of resolution and permit the distinction of the different populations (geographic races, sub-species): control region for mtDNA replication, regions coding for cytochrome b or coding for mitochondrial RNAs (RNA 12S or RNA 16S).

From a technical point of view, studies have been carried out on the mtDNA of the Anatidae. In the Anatidae, mtDNA has been sequenced in its entirety for only a single wild species: Aythya americana; however, it has been sequenced on numerous occasions in the various regions described previously in domestic species, such as the mallard (Anas platyrhynchos), the Muscovy duck (Cairina moschata), the greylag goose (Anser anser), but also in wild species. The majority of these studies propose analysis of the genetic diversity of the species by comparing the sequences with each other (Liu et al., 1996, Comp. Biochem. Physiol. 115B (2): 209-214). Other studies are based on a precise region of the mtDNA and calculate rates of divergence in order to study the evolution of one species compared with another (Ramirez et al., 1993, Journal of Molecular Evolution, 37,: 296-310). It is possible also to retrace the evolution of a species (Quinn et al., 1993, Journal of Molecular Evolution, 37: 417-425; Shield et al., 1987, Evolution, 41 (3): 662-666), and to add new elements regarding the place of a species on a phylogenic tree (Sraml et al., 1996, Autralian Journal of Zoology, 44: 47-58; Quinn, 1992, Molecular Ecololy, 1: 105-117, Johnson et al., 1998, Molecular Phylogenetics and Evolution, 10: 82-84).

Examination of the state of the art thus shows that programmes analysing the DNA of certain species of birds, in particular Anatidae, have already been carried out. However, none of the documents of the state of the art describes a specific and sensitive method of detecting and identifying the presence of biological materials derived from birds in a sample of organic material, applicable to all the genera of birds, and in particular species of birds, in samples of organic material presenting very varied compositions.

The applicant has therefore attempted to develop a sensitive and reliable method that allows this lack to be remedied.

One of the aims of the present invention is to provide a method of detecting the presence of biological materials derived from birds in a sample of organic material.

One of the other aims of the invention is to provide a method of identifying the genus, in particular the species of bird present in a sample of organic material.

Another aim of the invention is to provide a method permitting the distinction of species of birds that are phylogenetically close but have different commercial values.

Another aim of the invention is to provide a method of identifying the genus, in particular the species of bird present in fresh or processed (cooked, freeze-dried, dried, pickled, appertized etc.) food.

A subject of the present invention is a method of detecting the presence of biological materials derived from birds, in particular from Anseriformes, Galliformes or Struthioniformes, in a sample of organic material, characterized in that the presence of DNA derived from birds, in particular from Anseriformes, Galliformes or Struthioniformes, in the said organic material is determined by amplification of a DNA sequence specific to the genome of birds, in particular of the Anseriformes, Galliformes or Struthioniformes, and contained in the DNA extracted from the said sample, namely a sequence present in the genomes of birds, and in particular the Anseriformes, Galliformes or Struthioniformes but absent from the genomes of other animal genera, and in particular of other animal species.

A subject of the present invention is moreover a method of detecting the presence of biological materials derived from birds, and in particular from Anseriformes; Galliformes or Struthioniformes, in a sample of organic material and of identifying the genus, in particular the species of bird, in particular Anseriforme, Galliforme or Struthioniforme present in the said sample, characterized in that the presence of DNA derived from birds, and in particular from Anseriformes, Galliformes or Struthioniformes, in the said organic material is determined by amplification of a DNA sequence specific to the genome of birds, and in particular of the Anseriformes, Galliformes or Struthioniformes, and contained in the DNA extracted from the said sample, namely a sequence present in the genomes of birds, and in particular the Anseriformes, Galliformes or Struthioniformes, but absent from the genomes of other animal genera, in particular of other animal species, and in that the DNA sequence specific to the genome of birds, in particular of the Anseriformes, Galliformes or Struthioniformes, thus amplified, is compared with other DNA sequences of the genome of birds, and in particular of the Anseriformes, Galliformes or Struthioniformes, the said DNA sequence specific to the genome of birds, in particular of the Anseriformes, Galliformes or Struthioniformes, thus amplified, presenting at least 50% identity, in particular 60% identity with the other aforementioned DNA sequences of the genome of birds, and in particular of the Anseriformes, Galliformes or Struthioniformes.

By genus is meant an obligatory category to which every species must belong and which contains a species or a group of species (Wily, 1981).

By species is meant a population group really or partially capable of crossing and which is reproductively isolated from the other groups having the same property.

The animal species is divided into sub-species, races, varieties and strains; several neighbouring species form a genus which is itself a sub-division of the family.

By organic material is meant any solid or liquid material that is presumed to have at least partially an organic origin.

The percentage identity relates to the result of the comparison of the nucleic acids of the DNA sequence the identification of which is sought with those of the known DNA sequences of the birds, in particular the Anseriformes, the Galliformes or the Struthioniformes.

Thus, when the amplified DNA sequence specific to the genome of the birds presents at least 50% identity, in particular 60% with the other known DNA sequences of the genome of the birds, it will be possible to deduce from this that the sample of organic material to be analysed contains biological materials derived from birds, in particular from Anseriformes, Galliformes or Struthioniformes.

If the amplified DNA sequence specific to the genome of the birds presents less than 50% identity with the other known DNA sequences of the genome of the birds, it will thus be possible to deduce from this that the sample of organic material to be analysed does not contain biological materials derived from birds, in particular from Anseriformes, Galliformes or Struthioniformes.

According to an advantageous embodiment of the invention, the method permits the detection and/or identification of the presence of Anseriformes, in particular of Anatidae chosen from the group constituted by the ducks such as the pintail (Anas acuta), mandarin duck (Aix galericulata), shoveler (Anas clypeata), garganey (Anas querquedula), eider (Somateria mollissima), geese such as esser white-fronted goose (Anser erythropus), snow goose (Anser caerulescens), Canada goose (Branta canadensis), and swans such as the whooper swan (Cygnus cygnus), black swan (Cygnus atratus), mute swan (Cygnus olor) and in particular the domestic species of ducks such as the mallard (Anas platyrhynchos), Muscovy duck (Cairina moschata) or the domestic species of goose such as the greylag goose (Anser anser).

According to another advantageous embodiment of the invention, the method permits the detection and/or identification of the presence of Galliformes, in particular Gallinaceans chosen from the group constituted by chickens, quails and turkeys, and in particular species such as Gallus gallus, Coturnix coturnix or Meleagris gallopavo.

According to another advantageous embodiment, the method permits the detection and/or identification of the presence of Struthioniformes, in particular struthionidae such as ostriches, and in particular species such as Struthio cainelus.

Advantageously, the amplified sequence of the genome of the birds, in particular of Anseriformes, Galliformes or Struthioniformes, is of mitochondrial origin.

The choice of a mitochondrial sequence is particularly advantageous as in one animal cell there are approximately 100 to 1000 copies of mitochondrial DNA for one copy of nuclear DNA. In the event of degradation of the DNA, the probability of detecting mitochondrial DNA is therefore much greater than the probability of detecting nuclear DNA. Mitochondrial DNA can therefore be more surely detected in organic materials in which the DNA is subject to various physical (temperature, pressure, etc.) chemical or biochemical factors tending to its degradation.

According to an advantageous embodiment of the method of the invention, the DNA extracted from the sample of organic material is:
non-degraded DNA derived in particular from a fresh sample or,
degraded DNA, derived in particular from a processed, in particular cooked, freeze-dried, dried, pickled, appertized or pasteurized, sample.

Preferably, the amplification of the DNA sequence specific to the genome of birds, in particular of Anseriformes, Galliformes or Struthioniformes, is carried out by the polymerase chain (PCR) amplification method, comprising a repetition of the cycle of the following steps:
heating of the DNA extracted from the sample of organic material, so as to separate the DNA into two single-stranded strands,
hybridization of oligonucleotide primers as defined below to the single-stranded DNA strands at an adequate temperature and,
elongation of the said oligonucleotide primers by a polymerase at an adequate temperature, in order to obtain an amplified DNA sequence or DNA fragment specific to the genome of birds, in particular of Anseriformes, Galliformes or Struthioniformes.

In the following, the oligonucleotide primers can also be called “oligonucleotides” or “primers”. The amplified DNA sequence obtained at the end of the chain polymerization reaction (PCR) using the primers of the invention can also be called “amplified DNA fragment”, “DNA fragment” or “amplification product” in the following.

A subject of the invention is also a method of obtaining a DNA fragment derived from birds, in particular from Anseriformes and in particular from Anatidae, presenting a determined size and sequence, that is specific to the Anatidae, in particular to the genera Anas, Cairina and Anser, and in particular to the species Anas platyrhynchos, Cairina moschata and Anser anser, starting from a sample of organic material, a method by which there is amplified, by chain polymerisation reaction (PCR), a determined sequence of the genome of Anatidae that is present in the genomes of the Anatidae but absent from the genomes of the other animal species.

A subject of the invention is also a method of obtaining a DNA fragment derived from birds, especially from Galliformes and in particular from Gallinaceans, presenting a determined size and sequence, that is specific to the Gallinaceans, in particular to the genera Gallus, Coturnix and Meleagris, and in particular to the species Gallus gallus, Coturnix coturnix and Meleagris gallopavo, starting from a sample of organic material, a method by which there is amplified, by chain polymerisation reaction (PCR), a determined sequence of the genome of Gallinaceans that is present in the genomes of the Gallinaceans but absent from the genomes of the other animal genera or species.

A subject of the invention is also a method of obtaining a DNA fragment derived from birds, especially from Struthioniformes, and in particular from Struthionidae, presenting a determined size and sequence, that is specific to the Struthionidae, in particular to the genus Struthio, and in particular to the species Struthio camelus, starting from a sample of organic material, a method by which there is amplified, by chain polymerisation reaction (PCR), a determined sequence of the genome of Struthionidae that is present in the genomes of the Struthionidae but absent from the genomes of the other species.

According to an advantageous embodiment of the method of the invention, the amplified sequence of the genome of the birds, in particular of the Anseriformes, Galliformes or Struthioniformes, is situated in the central part of the control region for mitochondrial DNA replication, delimited by the nucleotides situated in the vicinity of positions 440 and 750, and in particular in the vicinity of positions 450 and 700, and preferably in the vicinity of positions 459 and 665 of the control region for mitochondrial DNA replication, the said positions being defined according to Desjardins and Morais, 1990, J. Mol. Biol., 599-634.

According to another advantageous embodiment of the method of the invention, the amplified sequence of the genome of the birds, in particular of the Anseriformes, Galliformes or Struthioniformes, is situated in the 5′ part of the control region for mitochondrial DNA replication, delimited by the nucleotides situated in the vicinity of positions 1 and 439, and in particular in the vicinity of positions 80 and 433, and preferably in the vicinity of positions 150 and 433 of the control region for mitochondrial DNA replication, or in the 3′ part of the control region for mitochondrial DNA replication, delimited by the nucleotides situated in the vicinity of positions 750 and 1227, and in particular in the vicinity of positions 780 and 1227, and preferably in the vicinity of positions 800 and 1227 of the control region for mitochondrial DNA replication, the said positions being defined according to Desjardins and Morais, 1990, J. Mol. Biol., 599-634.

A subject of the invention is also the oligonucleotides chosen from those:

(1)—presenting a sequence identity of at least 80%, preferably 90% and advantageously 95% with an oligonucleotide constituted by a sequence of approximately 15 to 25 nucleotides, in particular 20 to 25 nucleotides, contained in the following sequence SEQ ID No32 (positions 455 to 479 according to Desjardins and Morais, 1990, J. Mol. Biol., 212, 599-634):

ACG TGA CYA GCY TCA GGC CCA TAC G

in which Y is C or T,

or comprising the following sequence SEQ ID No33 (positions 465 to 479 according to Desjardins and Morais, 1990, J. Mol. Biol., 212, 599-634):

CTT CAG GCC CAT ACG

or constituted by the following sequence SEQ ID No1 (positions 459 to 479 according to Desjardins and Morais, 1990, J. Mol. Biol., 212, 599-634):

ACT AGC TTC AGG CCC ATA CG

or those,

(2)—presenting a sequence identity of at least 80%, preferably 90% and advantageously 95% with an oligonucleotide constituted by a sequence of approximately 15 to 25 nucleotides, in particular 20 to 25 nucleotides, contained in the following sequence SEQ ID No34 (positions 484 to 508 according to Desjardins and Morais, 1990, J. Mol. Biol., 212, 599-634):

CCC TAA ACC CCT CGC CCT CCT CAC A

or comprising the following sequence SEQ ID No35 (positions 493 to 508 according to Desjardins and Morais, 1990, J. Mol. Biol., 212, 599-634):

CTC GCC CTC CTC ACA

or constituted by the following sequence SEQ ID No2 (positions 488 to 508 according to Desjardins and Morais, 1990, J. Mol. Biol., 212, 599-634):

AAC CCC TCG CCC TCC TCA CA

or those,

(3)—presenting a sequence identity of at least &0%, preferably 90% and advantageously 95% with an oligonucleotide constituted by a sequence of approximately 15 to 25 nucleotides, in particular 20 to 25 nucleotides, contained in the following sequence SEQ ID No36 (positions 537 to 561 according to Desjardins and Morais, 1990, J. Mol. Biol., 212, 599-634):

GGG CCA TCM ATT GGG TTC ACT CAC C

in which M is A or C,

or comprising the following sequence SEQ ID No37 (positions 547 to 561 according to Desjardins and Morais, 1990, J. Mol. Biol., 212, 599-634):

TTG GGT TCA CTC ACC

or constituted by the following sequence SEQ ID No3 (positions 542 to 561 according to Desjardins and Morais, 1990, J. Mol. Biol., 212, 599-634):

ATC AAT TGG GTT CAC TCA CC

or those,

(4)—presenting a sequence identity of at least 80%, preferably 90% and advantageously 95% with an oligonucleotide constituted by a sequence of approximately 15 to 25 nucleotides, in particular 20 to 25 nucleotides, contained in the following sequence SEQ ID No38 (positions 574 to 599 according to Desjardins and Morais, 1990, J. Mol. Biol., 212, 599-634):

GGT RRA VST MNT CCA CAG ATG CCA C
in which R is A or G, V is A, C or G, N is A, C, G
or T, S is C or G, M is A or C,

or comprising the following sequence SEQ ID No39 (positions 584 to 599 according to Desjardins and Morais, 1990, J. Mol. Biol., 212, 599-634):

TTC CAC AGA TGC CAC

or constituted by the following sequence SEQ ID No4 (positions 580 to 599 according to Desjardins and Morais, 1990, J. Mol. Biol., 212, 599-634):

AAG TAT TCC ACA GAT GCC AC

or those,

(5)—presenting a sequence identity of at least 80%, preferably 90% and advantageously 95% with an oligonucleotide constituted by a sequence of approximately 15 to 25 nucleotides, in particular 20 to 25 nucleotides, contained in the following sequence SEQ ID No40 (positions 639 to 664 according to Desjardins and Morais, 1990, J. Mol. Biol., 212, 599-634):

CMG GAA RAR ADA ARW RGA ACC AGA G
in which R is A or G, D is A, G or T, M is A or C,
W is A or T,

or comprising the following sequence SEQ ID No41 (positions 648 to 664 according to Desjardins and Morais, 1990, J. Mol. Biol., 212, 599-634):

A TAA AAA GAA CCA GAG

or constituted by the following sequence SEQ ID No5 (positions 645 to 664 according to Desjardins and Morais, 1990, J. Mol. Biol., 212, 599-634):

AAA AAT AAA AGG AAC CAG AG

or those,

(6)—presenting a sequence identity of at least 80%, preferably 90% and advantageously 95% with an oligonucleotide constituted by a sequence of approximately 15 to 25 nucleotides, in particular 20 to 25 nucleotides, contained in the following sequence SEQ ID No42 (positions 797 to 820 according to Desjardins and Morais, 1990, J. Mol. Biol., 212, 599-634):

WVK CAY MYY KRY ACT GAT GCA CTT T
in which Y is C or T, K is G or T, R is A or G, M
is A or C, W is A or T, V is A, C or G, M is A or
C,

or comprising the following sequence SEQ ID No43 (positions 807 to 820 according to Desjardins and Morais, 1990, J. Mol. Biol., 212, 599-634):

ACA CTG ATG CAC TTT

or constituted by the following sequence SEQ ID No11 (positions 802 to 820 according to Desjardins and Morais, 1990, J. Mol. Biol., 212, 599-634):

CCT TGA CAC TGA TGC ACT TT

or those,

(7)—presenting a sequence identity of at least 80%, preferably 90% and advantageously 95% with an oligonucleotide constituted by a sequence of approximately 15 to 25 nucleotides, in particular 20 to 25 nucleotides, contained in the following sequence SEQ ID No44 (positions 1220 to 1224 according to Desjardins and Morais, 1990, J. Mol. Biol., 212, 599-634):

GCT TTD DND KKR WRS WAT GTG GAC G
in which Y is C or T, D is A, G or T, B is C, G or
T, K is G or T, W is A or T, N is A, C, G or T, R
is A or G, and S is C or G,

or comprising the following sequence SEQ ID No45 (positions 1210 to 1224 according to Desjardins and Morais, 1990, J. Mol. Biol., 212, 599-634):

TAA GCT ATG TGG ACG

or constituted by the following sequence SEQ ID No12 (positions 1205 to 1224 according to Desjardins and Morais, 1990, J. Mol. Biol., 212, 599-634):

TGT GGT AAG CTA TGT GGA CG

or those,

(8)—presenting a sequence identity of at least 80%, preferably 90% and advantageously 95% with an oligonucleotide constituted by a sequence of approximately 15 to 25 nucleotides, in particular 20 to 25 nucleotides, contained in the following sequence SEQ ID No46 (positions 180 to 206 according to Desjardins and Morais, 1990, J. Mol. Biol., 212, 599-634):

MTN MHM BDN HMV HVN NCA TAC CCT A
in which Y is C or T, M is A or C, H is A, C or T,
B is C, G or T, D is A, G or T, N is A, C, G or T,
W is A or T, V is A, C or G,

or comprising the following sequence SEQ ID No47 (positions 190 to 206 according to Desjardins and Morais, 1990, J. Mol. Biol., 212, 599-634):

AAC GGA CAT ACC CTA

or constituted by the following sequence SEQ ID No14 (positions 185 to 206 according to Desjardins and Morais, 1990, J. Mol. Biol., 212, 599-634):

CAT GTC AAC GGA CAT ACC CTA

or those,

(9)—presenting a sequence identity of at least 80%, preferably 90% and advantageously 95% with an oligonucleotide constituted by a sequence of approximately 15 to 25 nucleotides, in particular 20 to 25 nucleotides, contained in the following sequence SEQ ID No48 (positions 355 to 376 according to Desjardins and Morais, 1990, J. Mol. Biol., 212, 599-634):

CAT RAG NWA WKG KYG GGT GGA GAG G
in which R is A or G, N is A, C, G or T, W is A or
T, K is G or T, Y is C or T,

or comprising the following sequence SEQ ID No49 (positions 365 to 376 according to Desjardins and Morais, 1990, J. Mol. Biol., 212, 599-634):

GGG TGG GTG GAG AGG

or constituted by the following sequence SEQ ID No15 (positions 361 to 376 according to Desjardins and Morais, 1990, J. Mol. Biol., 212, 599-634):

TAA TGG GTG GGT GGA GAG G

or those,

(10)—presenting a sequence identity of at least 80%, preferably 90% and advantageously 95% with an oligonucleotide constituted by a sequence of approximately 15 to 25 nucleotides, in particular 20 to 25 nucleotides, contained in the following sequence SEQ ID No50 (positions 833 to 855 according to Desjardins and Morais, 1990, J. Mol. Biol., 212, 599-634):

GTT AWK GYY MYN CTC CGC RGC CCT T
in which W is A or T, K is G or T, Y is C or T, M
is A or C, Y is C or T, N is A, C, G or T, R is A
or G,

or comprising the following sequence SEQ ID No51 (positions 843 to 855 according to Desjardins and Morais, 1990, J. Mol. Biol., 212, 599-634):

TCC TCC GCA GCC CTT

or constituted by the following sequence SEQ ID No16 (positions 837 to 855 according to Desjardins and Morais, 1990, J. Mol. Biol., 212, 599-634):

ATG CTC TCC TCC GCA GCC CTT

or those,

(11)—presenting a sequence identity of at least 80%, preferably 90% and advantageously 95% with an oligonucleotide constituted by a sequence of approximately 15 to 25 nucleotides, in particular 20 to 25 nucleotides, contained in the following sequence SEQ ID No52 (positions 1120 to 1150 according to Desjardins and Morais, 1990, J. Mol. Biol., 212, 599-634):

DTW ATG ATT HTY RTT ADA TTA CGC T
in which D is A, G or T, W is A or T, H is A, C or
T, Y is C or T, R is A or G,

or comprising the following sequence SEQ ID No53 (positions 1130 to 1150 according to Desjardins and Morais, 1990, J. Mol. Biol., 212, 599-634):

TCG TTA GAT TAC GCT

or constituted by the following sequence SEQ ID No17 (positions 1125 to 1150 according to Desjardins and Morais, 1990, J. Mol. Biol., 212, 599-634):

GAT TGT CGT TAG ATT ACG CT

or those,

(12)—presenting a sequence identity of at least 80%, preferably 90% and advantageously 95% with an oligonucleotide constituted by a sequence of approximately 15 to 25 nucleotides, in particular 20 to 25 nucleotides, contained in the following sequence SEQ ID No54 (positions 249 to 272 according to Desjardins and Morais, 1990, J. Mol. Biol., 212, 599-634):

AAY GHA TGA AYG HYC AGN DAC CAT
in which Y is C or T, H is A, C or T, N is A, C, G
or T, D is A, G or T,

or comprising the following sequence SEQ ID No55 (positions 256 to 272 according to Desjardins and Morais, 1990, J. Mol. Biol., 212, 599-634):

ATG TTC TAG GAC CAT

or constituted by the following sequence SEQ ID No18 (positions 252 to 272 according to Desjardins and Morais, 1990, J. Mol. Biol., 212, 599-634):

ATG AAT GTT CTA GGA CCA T

or those,

(13)—presenting a sequence identity of at least 80%, preferably 90% and advantageously 95% with an oligonucleotide constituted by a sequence of approximately 15 to 25 nucleotides, in particular 20 to 25 nucleotides, contained in the following sequence SEQ ID No56 (positions 409 to 433 according to Desjardins and Morais, 1990, J. Mol. Biol., 212, 599-634):

CGG GTT CTG ATT TCA CGT GAG GAG T

or comprising the following sequence SEQ ID No57 (positions 419 to 433 according to Desjardins and Morais, 1990, J. Mol. Biol., 212, 599-634):

TTT CAC GTG AGG AGT

or constituted by the following sequence SEQ ID No19 (positions 414 to 433 according to Desjardins and Morais, 1990, J. Mol. Biol., 212, 599-634):

TCT GAT TTC ACG TGA GGA GT.

The oligonucleotides or primers as defined above permit the detection and identification of the DNA fragments derived from birds belonging to the order of the Anseriformes, and in particular to the family of the Anatidae, or to the order of the Galliformes and in particular to the family of the Gallinaceans.

A subject of the invention is also the primer pairs constituted:
by any one of the oligonucleotides SEQ ID No1, SEQ ID No32, SEQ ID No33, SEQ ID No2, SEQ ID No34, SEQ ID No35, SEQ ID No3, SEQ ID No36, SEQ ID No37 and any one of the oligonucleotides SEQ ID No4, SEQ ID No38, SEQ ID No39, SEQ ID No5, SEQ ID No40, SEQ ID No41 as defined above,
by any one of the oligonucleotides SEQ ID No11, SEQ ID No42, SEQ ID No43 and any one of the oligonucleotides SEQ ID No12, SEQ ID No44, SEQ ID No45 as defined above,
by any one of the oligonucleotides SEQ ID No14, SEQ ID No46, SEQ ID No47, and any one of the oligonucleotides SEQ ID No15, SEQ ID No48, SEQ ID No49 as defined above,
by any one of the oligonucleotides SEQ ID No16, SEQ ID No50, SEQ ID No51, and any one of the oligonucleotides SEQ ID No17, SEQ ID No52, SEQ ID No53 as defined above,
by any one of the oligonucleotides SEQ ID No18, SEQ ID No54, SEQ ID No55, and any one of the oligonucleotides SEQ ID No19, SEQ ID No56, SEQ ID No57 as defined above,
and advantageously constituted by the pair of oligonucleotides chosen from the following pairs:
SEQ ID No 1 and SEQ ID N 5,
SEQ ID No 2 and SEQ ID No 5,
SEQ ID No 1 and SEQ ID No 4,
SEQ ID No 2 and SEQ ID No 4,
SEQ ID No 3 and SEQ ID No 5,
SEQ ID No 11 and SEQ ID No 12,
SEQ ID No 14 and SEQ ID No 15,
SEQ ID No 16 and SEQ ID No 17,
SEQ ID No 18 and SEQ ID No 19.

The primer pairs as defined above permit the obtaining, by PCR reaction, of a DNA fragment derived from birds belonging to the order of the Anseriformes, and in particular to the family of the Anatidae, presenting a determined size and sequence, that is specific to the Anatidae. Moreover, the pair of primers (SEQ ID No11, SEQ ID No 12) permits the obtaining by PCR reaction of a DNA fragment derived from birds belonging to the order of the Galliformes, and in particular to the family of the Gallinaceans, presenting a determined size and sequence, that is specific to the Gallinaceans.

A subject of the invention is also the oligonucleotides chosen from those:

(1)—presenting a sequence identity of at least 80%, preferably 90% and advantageously 95% with an oligonucleotide constituted by a sequence of approximately 15 to 25 nucleotides, in particular 20 to 25 nucleotides, contained in the following sequence SEQ ID No58 positions 145 to 170 according to Desjardins and Morais, 1990, J. Mol. Biol., 212, 599-634):

ATA TGK MVN CGG CAT TAA CCT ATA T
in which K is G or T, M is A or C, V is A, C or G,
N is A, C, G or T,

or comprising the following sequence SEQ ID No59 (positions 155 to 170 according to Desjardins and Morais, 1990, J. Mol. Biol., 212, 599-634):

GGC ATT AAC CTA TAT

or constituted by the following sequence SEQ ID No23 (positions 151 to 170 according to Desjardins and Morais, 1990, J. Mol. Biol., 212, 599-634):

ATA CGG GCA TTA ACC TAT AT

or those,

(2)—presenting a sequence identity of at least 80%, preferably 90% and advantageously 95% with an oligonucleotide constituted by a sequence of approximately 15 to 25 nucleotides, in particular 20 to 25 nucleotides, contained in the following sequence SEQ ID No60 (positions 356 to 380 according to Desjardins and Morais, 1990, J. Mol. Biol., 212, 599-634):

GCG AGY ATA ACC AAA TSG GTT ACA T

in which Y is C or T, S is C or G,

or comprising the following sequence SEQ ID No61 (positions 366 to 380 according to Desjardins and Morais, 1990, J. Mol. Biol., 212, 599-634):

CCA AAT GGG TTA CAT

or constituted by the following sequence SEQ ID No24 (positions 361 to 380 according to Desjardins and Morais, 1990, J. Mol. Biol., 212, 599-634):

CAT AAC CAA ATG GGT TAC AT

or those,

(3)—presenting a sequence identity of at least 80%, preferably 90% and advantageously 95% with an oligonucleotide constituted by a sequence of approximately 15 to 25 nucleotides, in particular 20 to 25 nucleotides, contained in the following sequence SEQ ID No62 (positions 1014 to 1037 according to Desjardins and Morais, 1990, J. Mol. Biol., 212, 599-634):

TAC ATA CAW ACY MWC CGC ACA AAT A
in which W is A or T, Y is C or T, M is A or C,

or comprising the following sequence SEQ ID No63 (positions 1024 to 1037 according to Desjardins and Morais, 1990, J. Mol. Biol., 212, 599-634):

CTC ACC GCA CAA ATA

or constituted by the following sequence SEQ ID No25 (positions 1019 to 1037 according to Desjardins and Morais, 1990, J. Mol. Biol., 212, 599-634):

ACA AAC TCA CCG CAC AAA TA

or those,

(4)—presenting a sequence identity of at least 80%, preferably 90% and advantageously 95% with an oligonucleotide constituted by a sequence of approximately 15 to 25 nucleotides, in particular 20 to 25 nucleotides, contained in the following sequence SEQ ID No64 (positions 1167 to 1192 according to Desjardins and Morais, 1990, J. Mol. Biol., 212, 599-634):

TGT AAR TAW CAA TAT AAA TAA TAT A
in which R is A or G, W is A or T,

or comprising the following sequence SEQ ID No65 (positions 1177 to 1192 according to

AAT ATA AAT AAT ATA

or constituted by the following sequence SEQ ID No26 (positions 1172 to 1191 according to Desjardins and Morais, 1990, J. Mol. Biol., 212, 599-634):

TTA ACA ATA TAA ATA ATA TA

or those,

(5)—presenting a sequence identity of at least 80%, preferably 90% and advantageously 95%, with an oligonucleotide constituted by a sequence of approximately 15 to 25 nucleotides, in particular 20 to 25 nucleotides, contained in the following sequence SEQ ID No70 (positions 684 to 709 according to Desjardins and Morais, 1990, J. Mol. Biol., 212, 599-634):

CCT TCM CAG TGM BKK YGS CRG RGT V
in which M is A or C, B is C, G or T, K is G or T,
Y is C or T, S is C or G, R is A or G, V is A, C
or G,

or comprising the following sequence SEQ ID No171 (positions 694 to 709 according to Desjardins and Morais, 1990, J. Mol. Biol., 212, 599-634):

GCC GTC GCC AGA GTA

or constituted by the following sequence SEQ ID No72 (positions 689 to 709 according to Desjardins and Morais, 1990, J. Mol. Biol., 212, 599-634):

ACA GTG CCG TCG CCA GAG TA

or those,

(6)—presenting a sequence identity of at least 80%, preferably 90% and advantageously 95%, with an oligonucleotide constituted by a sequence of approximately 15 to 25 nucleotides, in particular 20 to 25 nucleotides, contained in the following sequence SEQ ID No 73 (positions 898 to 923 according to Desjardins and Morais, 1990, J. Mol. Biol., 212, 599-634):

RRW WRT RGW KGR RGK GRR WWT TNR Y
in which R is A or G, W is A or T, K is G or T, N
is A, C, G or T, Y is C or T,

or comprising the following sequence SEQ ID No74 (positions 898 to 913 according to Desjardins and Morais, 1990, J. Mol. Biol., 212, 599-634)

GAA GTG AAA ATT AGC

or constituted by the following sequence SEQ ID No75 (positions 898 to 918 according to Desjardins and Morais, 1990, J. Mol. Biol., 212, 599-634)

TAG AGG AAG TGA AAA TTA GC

or those,

(7)—presenting a sequence identity of at least 80%, preferably 90% and advantageously 95% with an oligonucleotide constituted by a sequence of approximately 15 to 25 nucleotides, in particular 20 to 25 nucleotides, contained in the following sequence SEQ ID No66 (positions 879 to 898 according to Desjardins and Morais, 1990, J. Mol. Biol., 212, 599-634):

TGA ATG CTT GYY GGA CAT WAA TAC C
in which Y is C or T, W is A or T,

or comprising the following sequence SEQ ID No67 (positions 888 to 898 according to Desjardins and Morais, 1990, J. Mol. Biol., 212, 599-634):

CTG GAC ATA AAT ACC

or constituted by the following sequence SEQ ID No27 (positions 884 to 898 according to Desjardins and Morais, 1990, J. Mol. Biol., 212, 599-634):

GCT TGC TGG ACA TAA ATA CC

or those,

(8)—presenting a sequence identity of at least 80%, preferably 90% and advantageously 95% with an oligonucleotide constituted by a sequence of approximately 15 to 25 nucleotides, in particular 20 to 25 nucleotides, contained in the following sequence SEQ ID No68 (positions 1077 to 1101 according to Desjardins and Morais, 1990, J. Mol. Biol., 212, 599-634):

CTA TAA TAK GRT GTG GGG CGT GTT G
in which K is G or T, R is A or G

or comprising the following sequence SEQ ID No69 (positions 1087 to 1101 according to Desjardins and Morais, 1990, J. Mol. Biol., 212, 599-634):

TTG TGG GGC GTG TTG

or constituted by the following sequence SEQ ID No28 (positions 1082 to 1101 according to Desjardins and Morais, 1990, J. Mol. Biol., 212, 599-634):

ATA AGT TGT GGG GCG TGT TG

The oligonucleotides or primers as defined above permit the detection and identification of the DNA fragments derived from birds belonging to the order of the Galliformes, and in particular to the family of the Gallinaceans, or to the order of the Struthioniformes, and in particular to the family of the Struthionides.

A subject of the invention is also the primer pairs constituted:
by any one of the oligonucleotides SEQ ID No23, SEQ ID No58, SEQ ID No59, and any one of the oligonucleotides SEQ ID No24, SEQ ID No60, SEQ ID No61 as defined above or,
by any one of the oligonucleotides SEQ ID) No25, SEQ ID No62, SEQ ID No63, and any one of the oligonucleotides SEQ ID No26, SEQ ID No64, SEQ ID. No65 as defined above or,
by any one of the oligonucleotides SEQ ID No70, SEQ ID No71, SEQ ID No72, and any one of the oligonucleotides SEQ ID No73, SEQ ID No074, SEQ ID No75 as defined above or,
by any one of the oligonucleotides SEQ ID No27, SEQ ID No66, SEQ ID No67, and any one of the oligonucleotides SEQ. ID No28, SEQ ID No68, SEQ ID No69 as defined above or,
and advantageously constituted by the pair of oligonucleotides chosen from the following pairs:
SEQ ID No23 and SEQ ID No24,
SEQ ID No25 and SEQ ID No26,
SEQ ID No72 and SEQ ID No75,
SEQ ID No27 and SEQ ID No28.

The primer pairs (SEQ ID No23, SEQ ID No24), (SEQ ID No25, SEQ ID No26) and (SEQ ID No72, SEQ ID No75), as defined above permit the obtaining, by PCR reaction, of a DNA fragment derived from birds belonging to the order of the Galliformes, and in particular to the family of the Gallinaceans, presenting a determined size and sequence, that is specific to the Gallinaceans.

The pair of primers (SEQ ID No27, SEQ ID No28) as defined above allows the obtaining, by PCR reaction, of a DNA fragment derived from birds belonging to the order of the Struthioniformes, and in particular to the family of the Struthionides, presenting a determined size and sequence, that is specific to the Struthionides.

A subject of the invention is also the DNA fragments that can be obtained according to the method as defined above, comprising roughly 100 to roughly 500 base pairs.

The DNA fragment of the invention advantageously presents a sequence identity of at least 80%, preferably 90% and advantageously 95% with:

an oligonucleotide constituted by a sequence of approximately 15 to 25 nucleotides, contained in the following SEQ ID No6:

ACYAGCTTCA GGCCCATACG TTCCCCCTAA ACCCCTCGCC
CTCCTCACAT TTTTGCGCCT CTGGTTCCTC GGTCAGGGCC
ATCMATTGGG TTCACTCACC YCYMYTYGCC YTTCAAAGTG
GCATCTGTGG ANKACBTYCA CCWYYYCRRT GCGTWATCGC
GGCATBYTYM ASYWTTTWSM CGCCTYTGGT TCYMYTTHTY
TYT

in which Y is C or T, M is A or C, K is G or T, B is C, G or T, R is A or G, W is A or T, S is C or G, H is A, C or T, N is A, C, G or T,
and in particular a fragment of 203 base pairs comprising or constituted by the SEQ ID No6 defined above,

or an oligonucleotide constituted by a sequence of approximately 15 to 25 nucleotides, contained in the following SEQ ID No7:

ACYAGCTTCA GGCCCATACG TTCCCCCTAA ACCCCTCGCC
CTCCTCACAT TTTTGCGCCT CTGGTTCCTC GGTCAGGGCC
ATCMATTGGG TTCACTCACC YCYMYTYGCC YTTCAAAGTG
GCATCTGTGG ANKACBT

in which Y is C or T, M is A or C, N is A, C, G or T, K is G or T, B is C, G or T,
and in particular a fragment of 137 base pairs comprising or constituted by the SEQ ID No7 defined above,

or an oligonucleotide constituted by a sequence of approximately 15 to 25 nucleotides, contained in the following SEQ ID No8:

AACCCCTCGC CCTCCTCACA TTTTTGCGCC TCTGGTTCCT
CGGTCAGGGC CATCMATTGG GTTCACTCAC CYCYMYTYGC
CYTTCAAAGT GGCATCTGTG GANKACBTYC ACCWYYYCRR
TGCGTWATCG CGGCATBYTY MASYWTTTWS MCGCCTYTGG
TTCYMYTTHT YTYT

in which Y is C or T, M is A or C, N is A, C, G or T, K is G or T, B is C, G or T, R is A or G, W is A or T, S is C or G, H is A, C or T,
and in particular a fragment of 174 base pairs comprising or constituted by the SEQ ID No8 defined above,

or an oligonucleotide constituted by a sequence of approximately 15 to 25 nucleotides, contained in the following SEQ ID No9:

AACCCCTCGC CCTCCTCACA TTTTTGCGCC TCTGGTTCCT
CGGTCAGGGC CATCMATTGG GTTCACTCAC CYCYMYTYGC
CYTTCAAAGT GGCATCTGTG GANKACBT

in which Y is C or T, M is A or C, N is A, C, G or T, K is G or T, B is C, G or T,
and in particular a fragment of 108 base pairs comprising or constituted by the SEQ ID No 9 defined above,
or an oligonucleotide constituted by a sequence of approximately 15 to 25 nucleotides, contained in the following SEQ ID No10:
   ATCMATTGGG TTCACTCACC YCYMYTYGCC YTTCAAAGTG GCATCTGTGG ANKACBTYCA CCWYYYCRRT GCGTWATCGC GGCATBYTYM ASYWTTTWSM CGCCTYTGGT TCYMYTTHTY TYT
in which Y is C or T, M is A or C, N is A, C, G or T, K is G or T, B is C, G or T, R is A or G, W is A or T, S is C or G, H is A, C or T,
and in particular a fragment of 123 base pairs comprising or constituted by the SEQ ID No 10 defined above,

or an oligonucleotide constituted by a sequence of approximately 15 to 25 nucleotides, contained in the following SEQ ID No13:

YMYYKRYACT GATGCACTTK GDYCDCATTY RGTTRWKGYY
MYNTCCRCHV HYCYABNBWH WVRTGYTRYY NRGTGAATGC
TYGHHGGACA TADYWYWAWM MAWHWWMHYW YYHWSYRYWW
YYYHYRSRMH HHMNRAAYDM AACCARHRDW YTTYMDCHRW
THWDHWWHMM WHYWYVTWDY RTMWWHYHHV HMHHHHMHWM
HHBHAWWHHH HNHNHYNHWA YYBCHNNYHH HWAWYNYSYY
WHHYNWBVHW HHNBYBDMDM DWCHDWMMNR WWHDMAYDDH
DYVDHHYAWM HTTATTAKAG RAACWCCAGY ACYARVVVHD
VWHNHAMHYN RRHRWHHMYY AYWAHYYHVH YNTYNMNAMH
HNBNVYHYRD HYHRYYARYB RMHYVHHHYR YYCDCMTWSY
WYMMHNHH

in which Y is C or T, M is A or C, N is A, C, G or T, K is G or T, B is C, G or T, R is A or G, W is A or T, S is C or G, D is A, G or T, V is A, C or G, H is A, C or T,
and in particular a fragment of 408 base pairs comprising or constituted by the SEQ ID No13 defined above or,
fragments resulting from one or more deletions of contiguous or non-contiguous nucleotides of the said fragment of 408 base pairs comprising or constituted by SEQ ID No13, and capable of being obtained by amplification of mtDNA derived from birds, using the oligonucleotides SEQ ID No11 and SEQ ID No12 as defined previously, and in particular fragments of 331 base pairs, 328 base pairs or 288 base pairs,

or an oligonucleotide constituted by a sequence of approximately 15 to 25 nucleotides, contained in the following SEQ ID No20:

HWBDNHMRMG RNCATHHHHN NYHVHBDNAH NNNHCHHCCA
VVRMHHHNNV RTGMAYGHYC YHRNDVHHHH NHVNHVVMHV
VMHVNVHNHN NHHHNHVHVV RWNMACMARR RCMMHDHNNR
VTGAATGCTH NMHRRACAWR DMNYNDVHAH HMHTCYDDNB
YYBHHTCCAC CCRMMCATWH

in which Y is C or T, M is A or C, N is A, C, G or T, B is C, G or T, R is A or G, W is A or T, D is A, G or T, V is A, C or G, H is A, C or T,
and in particular a fragment of 180 base pairs comprising or constituted by the SEQ ID No20 defined above,

or an oligonucleotide constituted by a sequence of approximately 15 to 25 nucleotides, contained in the following SEQ ID No21:

WKGYYMYNTC CRCHVHYCYA BNBWHWVRTG YTRYYNRGTG
AATGCTYGHH GGACATAMHH HMNRAAYDMA ACHRHYHHVH
MHHHNHNHNH HNNYHHHWAY WBVHWHWCHD WMMNRWWHDM
AYDDHDYVDH HYAWMHTTAT TAKAGRAACW CCAGYACYAR
VVVHDVWHNH AMHYNRRHRW HHMYYAYWAH Y

in which Y is C or T, M is A or C, N is A, C, G or T, B is C, G or T, R is A or G, W is A or T, D is A, G or T, V is A, C or G, H is A, C or T, K is G or T,
and in particular a fragment of 191 base pairs comprising or constituted by the SEQ ID N 21 defined-above,

or an oligonucleotide constituted by a sequence of approximately 15 to 25 nucleotides, contained in the following SEQ ID No22:

RTGAAYGHYC YHRNDVHHNH MVHNHNVMHV VNHNHHNHNH
VVVSWNMARD SCMNHNNTTM VTGAATGCTH NMHRRACAWD
MNYNDVHAHH MHTCYNDNBH YBHHTCCACC CRMMCATWHM
TYATGRAVYY NCGTAYCARR TGGATTTATT RRYCGTACWC
CTCACGTGAA ATCAGC

in which Y is C or T, M is A or C, N is A, C, G or T, B is C, G or T, R is A or G, W is A or T, D is A, G or T, V is A, C or G, H is A, C or T, S is C or G,
and in particular a fragment of 176 base pairs comprising or constituted by the SEQ ID No 22 defined above.

The DNA fragments as defined above are specific to birds, in particular to Anseriformes or Galliformes, and more particularly to Anatidae and Gallinaceans.

By way of example, the nucleotide sequence of the fragment SEQ ID No6 as defined below is specific to the mandarin duck (Aix galericulata) and is obtained by amplification using the primers SEQ ID No1 and SEQ ID. No5:

ACTAGCTTCA GGCCCATACG TTCCCCCTAA ACCCCTCGCC
CTCCTCACAT TTTTGCGCCT CTGGTTCCTC GGTCAGGGCC
ATCAATTGGG TTCACTCACC TCCTCCTTGC CTTTCAAAGT
GGCATCTGTG GAAGACCTCC ACCATCTCAA TGCGTAATCG
CGGCATCCTC CAGCTTTTTG GCGCCTCTGG TTCCTCTTAT
TTT

The invention also relates to the DNA fragment advantageously presenting a sequence identity of at least 80%, preferably 90% and advantageously 95% with:

an oligonucleotide constituted by a sequence of approximately 15 to 25 nucleotides, contained in the following SEQ ID No29:

KMAWVNGGHM WTVACHYATA TTCCMCWTYY CKYMCHAHVB
MCHYTVDATG MAYSMTSHHR KRCMHVCTMH NTTHCVYDHM
CCATAGABAG TTCCWAACHA CWATCRRKVM MCMTAACTAT
GAATGGTTRC VGHACATAAA TCTCACTCTC ATGTTCTCCC
CCCAACAAGT CACCTAACTA TGAATGGTTA CAGGACATAC
ATTTDACTAY CATGTTCTAA CCSATTTGGT T

in which Y is C or T, M is A or C, N is A, C, G or T, B is C, G or T, R is A or G, W is A or T, D is A, G or T, V is A, C or G, H is A, C or T, S is C or G, K is G or T
and in particular a fragment of 231 base pairs comprising or constituted by the SEQ ID No29 defined above,

or an oligonucleotide constituted by a sequence of approximately 15 to 25 nucleotides, contained in the following SEQ ID No30:

| | | | | | |
|---|---|---|---|---|---|
| TACAWACYMW | CGYAWWRRWD | CCCTYAMAYY | AYAHAAMCGY | YTATCGYWTA | DTATATATAC |
| ATTRYWRYDY | AYYMYAYMAT | TATTAKAGAA | ACYCCMCTAC | CRAAACMAWC | AWHARARRCW |
| WAHADHWAHA | TKCMRCDYAW | VBYHCYTCAS | AWRCMWWBRT | KRTWTATATT | GTTAA |

in which Y is C or T, M is A or C, B is C, G or T, R is A or G, W is A or T, D is A, G or T, V is A, C or G, H is A, C or T, S is C or G, K is G or T,
and in particular a fragment of 175 base pairs comprising or constituted by the SEQ ID No30 defined above,

or an oligonucleotide constituted by a sequence of approximately 15 to 25 nucleotides, contained in the following SEQ ID No76:

| | | | | | |
|---|---|---|---|---|---|
| MCAGTGMSKK | YGSCRGRGTV | CYAYTCAAKY | KRAGCCTGGA | YTACWCCTGC | GTTRCGYSCT |
| ATYCTAKWYY | TCHVGKRTYM | CTCRATGAKA | CGGTTKGCGT | RTATKKGGWA | TCAYYTTGAC |
| ACTGATGCAC | TTTGGAYCRC | ATTYRGTTAW | KGYTCTYCCR | CMSYYCYNKW | WARTRGKGYT |
| ATWTAGTGAA | TGCTYGHYGG | ACATAYYTTA | WSYNAAWWYY | CMCYYCMWCY | A |

in which M is A or C, S is C or G, K is G or T, Y is C or T, R is A or G, V is A, C or G, W is A or T, N is A, C, T or G,
and in particular a fragment of 231 base pairs comprising or constituted by the SEQ ID No76 defined above,

or an oligonucleotide constituted by a sequence of approximately 15 to 25 nucleotides, contained in the following SEQ ID No31:

| | | | | | |
|---|---|---|---|---|---|
| TGAATGCTTG | YYGGACATWW | TWYMAYYWAT | TWTCACTTCC | WCTGAYTTTY | YTMACAMMAC |
| YAGGAYWMAW | YTYYTWYDTY | DKTWYWAAYW | YWTKWTTTVW | TYAYWTYAAW | MACATYTTTT |
| AMYTAWAYTA | MCYWTACAWA | CYMWCMGYAY | WRRWTDTYAM | AYYAAHAAMC | GYYCGYWTAD |
| TATATATACA | TARYWRYTGY | RYYTMYAYCM | ATTATT | | |

in which Y is C or T, M is A or C, R is A or G, W is A or T, D is A, G or T, V is A, C or G, H is A, C or T,
and in particular a fragment of 216 base pairs comprising or constituted by the SEQ ID No31 defined above.

The DNA fragments SEQ ID No29, SEQ ID No30 and SEQ ID No76 as defined above are specific to birds belonging to the order of the Galliformes, and more particularly to the family of the Gallinaceans; the DNA fragment SEQ ID No31 as defined above is specific to birds belonging to the order of the Struthioniformes, and more particularly to the family of the Struthionides.

According to an advantageous embodiment of the method of the invention, the amplified DNA fragment obtained is identified:
by sequencing of the said amplified fragment,
by gel electrophoresis of the fragments obtained after digestion using restriction enzymes of the said fragment,
directly, by simple visualization of the presence of the said amplified fragment by gel electrophoresis.

According to an advantageous embodiment of the invention, the method of identification by sequencing of the amplified fragment obtained permits the identification of the species of birds belonging to the order of the Anseriformes, and more particularly to the family of the Anatidae.

The method of identification by gel electrophoresis of the fragments obtained after digestion using restriction enzymes permits identification of the genera of birds belonging to the order of the Anseriformes, and more particularly the family of the Anatidae, and/or to the order of the Galliformes, and more particularly the family of the Gallinaceans.

The method of direct identification, by simple visualization of the presence of the amplified sequence obtained, permits the identification of the species of birds belonging to the order of the Anseriformes, and more particularly to the family of the Anatidae, to the order of the Galliformes, and more particularly to the family of the Gallinaceans, and/or to the order of the Struthioniformes, and more particularly to the family of the Struthionides.

Each of these three methods of identification is described in more detail below.

1) The method of identification by sequencing of the amplified DNA sequence or fragment is called “global strategy” in the following, as it permits the detection and identification of the presence or the absence of all the existing Anatidae. The list of all the existing Anatidae that can be detected and identified by the method of the invention is given in table 1 hereafter.

The primers used in order to carry out the amplification by the PCR method of a DNA sequence derived from Anatidae, possibly present in a sample of organic material to be analysed, are the 15 primers listed below and as defined above:
SEQ ID No32, SEQ ID No33, SEQ ID No1, SEQ ID No34, SEQ ID No35, SEQ ID No2, SEQ ID No36, SEQ ID No37, SEQ ID No3, SEQ ID No 38, SEQ ID No39, SEQ ID No4, SEQ ID No40, SEQ ID No41, SEQ ID No5,
and more particularly the 5 primers SEQ ID No1, SEQ ID No2, SEQ ID No3, SEQ ID No4 and SEQ ID No5.

Each of the oligonucleotides SEQ ID No1 to SEQ ID No3 can be used paired with any one of the oligonucleotides SEQ ID No4 and SEQ ID No5.

According to a particularly advantageous mode of implementation of the present invention, part of the hybridization steps of the cycles constituting the amplification reaction is carried out at a temperature of approximately 50° C. to approximately 58° C. The Applicant also found that a temperature of 55° C. was particularly suitable for obtaining a specific amplification. Such a mode of implementation allows a greater specificity of amplification to be obtained.

It will be noted on this point that the Applicant has solved a certain number of technical problems in order to carry out this method. First of all, the choice of the primers constituted a real problem, as it was necessary to select on the one hand sequences common to the different species of Anatidae but not to other species of birds, and on the other hand hybridizing in a stable manner, in very variable physico-chemical conditions, representative of the great variability of the organic materials capable of containing biological materials derived from Anatidae. The temperatures of the steps of separation and elongation of strands are advantageously approximately 94° C. and approximately 72° C. respectively.

The method described above is specific to the Anatidae but global between all the species of Anatidae as it does not give an amplification reaction that can be detected in the presence of DNA of an origin other than the Anatidae.

According to an advantageous embodiment of the method of the invention, the use of the following oligonucleotide primer pairs
(SEQ ID No1, SEQ ID No5),
(SEQ ID No2, SEQ ID No5),
(SEQ ID No1, SEQ ID No4),
(SEQ ID No2, SEQ ID No4) and,
(SEQ ID) No3, SEQ ID No5),
permits the obtaining of, respectively:
a DNA fragment SEQ ID No6 of 203 base pairs as defined above,
a DNA fragment SEQ ID No8 of 174 base pairs as defined above,
a DNA fragment SEQ ID No7 of 137 base pairs as defined above,
a DNA fragment SEQ ID No9 of 108 base pairs as defined above and,
a DNA fragment SEQ ID No10 of 123 base pairs as defined above.

The amplification products described above can be detected even when a substantial fraction of the DNA is degraded, namely after action of physical, chemical and/or biochemical factors, and during various transformations of the samples of organic materials. The experimenter preferably searches for the presence of the sequence SEQ ID no6 described above using appropriate primers, and in the case where the detection would be negative, he searches for the fragments of small size and in particular those of approximately 174, 137, 123 and 108 base pairs.

The uniqueness of the amplification products is another advantage of the present invention, as this allows a substantial sensitivity to be achieved and greatly facilitates the interpretation of the results. The method according to the present invention presents a large number of advantages compared with the already known identification techniques.

Thus, the method described presents a great simplicity of interpretation, due to the production of a single and unique product, specific to the DNA of Anatidae and which therefore is not found in the DNA amplification products of other species.

The amplification product can be demonstrated by any method known to a person skilled in the art, and in particular by simple agarose gel electrophoresis. The reading of the migration profiles of the amplification products obtained with the method of the invention therefore simply involves the determination of the presence of a single and unique migration band in an electrophoresis gel. In the absence of such a band, it can be considered that there are no detectable traces of DNA of Anatidae. On the other hand, the presence of a band means that the DNA of Anatidae is present in the sample, and therefore that the sample in question contains biological material based on Anatidae.

The amplification product obtained can then be sequenced in order to determine its nucleotide sequence. The comparison of this sequence with all the known nucleotide sequences of the Anatidae permits determination of the species of Anatidae present in the sample of organic material, and thus differentiation of the species relative to one another.

The amplification product obtained, identified using the global strategy, is generally situated in the central part of the control region for mitochondrial DNA replication, delimited by the nucleotides situated in the vicinity of positions 440 and 750, in particular in the vicinity of positions 450 and 700, and preferably in the vicinity of positions 459 and 665 of the control region for mitochondrial DNA replication, the said positions being defined according to Desjardins and Morais, 1990, J. Mol. Biol., 599-634.

2) The method of identification by gel electrophoresis of the fragments obtained using restriction enzymes of the said amplified fragment is called “intermediate strategy”, as it permits the detection and identification of the most common genera of Anatidae and Gallinaceans.

A result can be achieved more rapidly with the intermediate strategy than with the “global strategy”, as the sequencing phase is avoided. This method of identification involves the placing of an amplification product obtained by PCR in the presence of restriction enzymes. The oligonucleotides are chosen so as to have a pretty variable amplification fragment in order to be able to sufficiently distinguish genera of Anatidae and Gallinaceans after enzymatic cleavage.

The primers used in order to carry out the amplification by the PCR method of a DNA sequence derived from Anatidae or Gallinaceans, possibly present in a sample of organic material to be analysed, are the 6 primers listed hereafter and as defined above: SEQ ID No42, SEQ ID No43, SEQ ID No11, SEQ ID No44, SEQ ID No45 and SEQ ID No12, and more particularly the 2 primers SEQ ID No11 and SEQ ID No12.

According to an advantageous embodiment of the method of the invention, the use of the pair of oligonucleotides SEQ ID No11 and SEQ ID No12 allows an amplification fragment SEQ ID No13 of a variable size to be obtained, according to the genus of Anatidae or Gallinaceans present in the sample of organic material to be analysed.

Thus, an amplification fragment SEQ ID No13 of a size of 408 bp, obtained using the pair (SEQ ID No11, SEQ ID No12), is characteristic of the goose of the genus Anser, and in particular of the species Anser anser (greylag goose). The nucleotide sequence of this fragment was defined above.

An amplification fragment SEQ ID No13 of a size of 288 bp, obtained using the pair (SEQ ID No11, SEQ ID No12), is characteristic of the duck of the genus Anas, and in particular of the species Anas platyrhynchos (mallard) or of the genus Cairina, and in particular of the species Cairina moschata (Muscovy duck), and presents the following nucleotide sequence:

| | | | | | |
|---|---|---|---|---|---|
| YMYYKRYACT | GATGCACTTK | GDYCDCATTY | RGTTRWKGYY | MYNTCCRCHV | HYCYABNBWH |
| WVRTGYTRYY | NRGTGAATGC | TYGHHGGACA | TAMHHHMNRA | AYDMAACHRH | YHHVHMHHHN |
| HNHYNHHNNY | HHHWAYWBVH | WHWCHDWMMN | RWWHDMAYDD | HDYVDHHYAW | MHTTATTAKA |
| GRAACWCCAG | YACYARVVVH | DVWHNHAMHY | NRRHRWHHMY | YAYWAHYYHV | HYNTYNMNMH |
| HNBNVYHYRD | HYHRYYARYB | RMHYVHHHYR | YYCDCMTWSY | WYMMHNHH | |

in which Y is C or T, M is A or C, N is A, C, G or T, K is G or T, B is C, G or T, R is A or G, W is A or T, S is C or G, D is A, G or T, V is A, C or G, H is A, C or T.

An amplification fragment SEQ ID No13 of a size of 328 bp, obtained using the pair (SEQ ID No11, SEQ ID No12), is characteristic of the swan of the genus Cygnus and in particular of the species Cygnus cygnus (whooper swan), and presents the following nucleotide sequence:

| | | | | | |
|---|---|---|---|---|---|
| YMYYKRYACT | GATGCACTTK | GDYCDCATTY | RGTTRWKGYY | MYNTCCRCHV | HYCYABNBWH |
| WVRTGYTRYY | NRGTGAATGC | TYGHHGGACA | TAMHHHMNRA | AYDMAACCAY | DMAACHRHYH |
| HVHMHHHNHN | HYNHHNNYRH | HWAYYBCHNN | YHHWAWYMYS | YYWHHYNWBV | HWHNBYBDMD |
| MDWCHDWMNR | WWHDMAYDDH | DYVDHHYAWM | HTTATTAKAG | RAACWCCAGY | ACYARVVVHD |
| VWHNHAMHYN | RRHRWHHMYY | AYWAHYYHVH | YNTYNMNAMH | HNBNVYHYRD | HYHRYYARYB |
| RMHYVHHHYR | YYCDCMTWSY | WYMMHNHH | | | |

in which Y is C or T, M is A or C, N is A, C, G or T, K is G or T, B is C, G or T, R is A or G, W is A or T, S is C or G, D is A, G or T, V is A, C or G, H is A, C or T.

A fragment SEQ ID No13 of a size of 331 bp, obtained using the pair (SEQ ID No11, SEQ ID No12), is characteristic of the chicken of the genus Gallus, and in particular of the species Gallus gallus jungle fowl) and presents the following nulceotide sequence:

| | | | | | |
|---|---|---|---|---|---|
| YMYYKRCTGA | TGCACTTKGD | YCDCATTYRG | TTRWKGYYMY | CCRCHVHYCY | ABNBWHWVRT |
| GYTRYYNRGT | GAATGCTYGH | HGGACATADY | WYWAWMMAWH | WMHYWYYHWS | YRYWWYYYHY |
| RSRMHHHMRA | AYDMAACCAR | HRDWYTTYMD | CHRWTHDYRT | MWWHYHVHMH | HNHWAYYBCH |
| NNYHDMDWCH | DWMMRWWHDM | AYDDHDYVDH | HYAWHTTATT | AKAGRAACWC | CAGYACYARV |
| VVHDVWHNHA | MHYNRRHRWH | MYYAYWAHYY | HVHYNTYNMN | AMHHNBNVYH | YRDHYHRYYA |
| RYBRMHYVHH | HYRYYCDCMT | WSYWYMMHNH | H | | |

in which Y is C or T, M is A or C, N is A, C, G or T, K is G or T, B is C, G or T, R is A or G, W is A or T, S is C or G, D is A, G or T, V is A, C or G, H is A, C or T.

In fact, the primers SEQ ID No11 and SEQ ID No12 frame a part of the mtDNA control region which varies by virtue of a DNA deletion respectively in the duck, the swan and the chicken compared with the goose.

According to a particularly advantageous mode of implementation of the present invention, part of the hybridization steps of the cycles constituting the amplification reaction is carried out at a temperature of approximately 50° C. to approximately 58° C. The applicant also found that a temperature of 55° C. was particularly suitable for obtaining a specific amplification. Such a mode of implementation allows a greater specificity of amplification to be achieved.

The amplified fragment SEQ ID No13 obtained is subjected to various restriction enzymes specific to different genera of birds of the family of the Anatidae or the Gallinaceans. Thee enzymes were chosen in order to have different cleavage sites on the sequences of the genera chosen.

By way of example there can be cited, when it is a matter of identifying an amplified DNA sequence derived from Anatidae, the restriction enzymes chosen from the group constituted by Nsi I, Acc I, Hha I or Nde I or their mixtures.

The enzymes Nsi I, Acc I, Hia I and Nde I are specific to the genera of Anatidae most often found in samples of organic material. More particularly, enzyme Nsi I is specific to the genus Anas, including in particular the species Anas platyrhynchos (pintail), Anas acuta (mallard), Anas querquedula (garganey); the enzyme Acc I is specific to the genus Cairina, including in particular the species Cairina moschata (Muscovy duck); the enzyme Hha I is specific to the genus Anser, including in particular the species Anser anser (greylag goose), Anser caerulescens (snow goose); the enzyme Nde I is specific to the genus Cygnus including in particular the species Cygnus Cygnus (whooper swan), Cygnus atratus (black swan).

When it is a matter of identifying an amplified DNA sequence derived from Gallinaceans, the restriction enzyme BsaJ I will be used.

The enzyme BsaJ I is specific to the genus of Gallinaceans that is most frequently found in a sample of organic material, namely the genus Gallus, including in particular the species Gallus gallus (chicken).

Thus, according to the size of the fragments obtained using the aforementioned restriction enzymes, the species of Anatidae or of Gallinaceans present in the sample of organic material is determined, and the species can thus be distinguished from one another.

By way of example, using the enzyme Nsi I, two fragments of a size of 154 bp and 134 bp respectively, characteristic of the genus Anas, are obtained. The enzyme Acc I leads to two fragments of a size of 147 bp and 141 bp respectively, characteristic of the genus Cairina. The enzyme Bha I leads to three fragments of a size of 11 bp, 113 bp and 284 bp respectively, characteristic of the genus Anser. The enzyme Nde I leads to two fragments of a size of 132 bp and 196 bp respectively, characteristic of the genus Cygnus. The enzyme BsaJ I leads to two fragments of a size of 50 bp and 281 bp, characteristic of the genus Gallus.

3) The method of direct identification, by simple visualization of the presence of the amplified DNA fragment using a gel electrophoresis, is called “specific strategy” in the following, as its permits the direct detection and identification of the very specific species of Anatidae, Gallinaceans and Struthionides. In the “specific strategy”, the detection and identification step is a simultaneous step.

In fact, this method, unlike the two methods described above, does not require an additional step after the obtaining of the amplified sequence and thus permits the detection and identification directly after amplification of certain very particular species of birds, which are the domestic species that are the most common as they are the most used in food or other preparations. These specific species of birds that can be directly detected and identified are:
the mallard or common duck (Anas platyrhynchos), the Muscovy duck (Cairina moschata) and the greylag goose (Anser anser) for the birds belonging to the family of the Anatidae (order of the Anseriformes),
the chicken (Gallus gallus), the quail (Coturnix coturnix) and the turkey (Meleagris gallopavo) for the birds belonging to the family of the Gallinaceans (order of the Galliformes),
the ostrich (Struthio camelus) for the birds belonging to the family of the Struthionides (order of the Struthioniformes).

The amplification of the DNA is carried out by the chain polymerase (PCR) amplification method as described previously.

The specific strategy permitting the detection and identification of the presence of Anatidae in a sample of organic material is described in more detail below.

a) Specific Strategy Permitting the Identification of the Anatidae.

The primers used in order to carry out the amplification by the PCR method of a DNA sequence derived from Anatidae, possibly present in a sample of organic material to be analysed, are the 18 primers listed below and as defined above:
SEQ ID No46, SEQ ID No47, SEQ ID No14, SEQ ID No48, SEQ ID No49, SEQ ID No15, SEQ ID No50, SEQ ID No51, SEQ ID No16, SEQ ID No52, SEQ ID No53, SEQ ID No17, SEQ ID No54, SEQ ID No55, SEQ ID No18, SEQ ID No56, SEQ ID No57 and SEQ ID No19,
and more particularly the 6 primers SEQ ID No14, SEQ ID No15, SEQ ID No16, SEQ ID No17, SEQ ID No18 and SEQ ID No9.

According to an advantageous embodiment of the method of the invention, the use of the following pairs of oligonucleotides:
(SEQ ID No14, SEQ ID No15),
(SEQ ID No16, SEQ ID No17) and
(SEQ ID No18, SEQ ID No19),
permits the obtaining of, respectively:
a fragment SEQ ID No20 of 180 base pairs as defined above,
a fragment SEQ ID No21 of 191 base pairs as defined above, and
a fragment SEQ ID No22 of 176 base pairs as defined above.

According to a particularly advantageous mode of implementation of the present invention, part of the hybridization steps of the cycles constituting the amplification reaction is carried out at a temperature of 63° C. which is particularly suitable for obtaining a specific amplification. Such a mode of implementation allows a greater specificity of amplification to be obtained.

The presence of the fragments SEQ ID No20, SEQ ID No21 and SEQ ID No22 after amplification is detected by simple visualization by agarose gel electrophoresis.

The visualization of the fragment SEQ ID No20 permits the detection and identification of the presence of mallard (Anas platyrhynchos). The visualization of the fragment SEQ ID No21 permits the detection and identification of the presence of Muscovy duck (Cairina moschata). The visualization of the fragment SEQ ID No22 permits the detection and identification of the presence of greylag goose (Anser anser).

The specific strategy permitting the detection and identification of the presence of Gallinaceans or of Struthionides in a sample of organic material is described in more detail below.

b) Specific Strategy Permitting the Identification of the Gallinaceans or the Struthionides.

The primers used in order to effect the amplification by the PCR method of a DNA sequence derived from Galliformes, possibly present in a sample of organic material to be analysed, are the 12 primers listed below and as defined above:
SEQ ID No58, SEQ ID No59, SEQ ID No23, SEQ ID No60, SEQ ID No61, SEQ ID No24, SEQ ID No62, SEQ ID No63, SEQ ID No25, SEQ ID No64, SEQ ID No65, SEQ ID No26, SEQ ID No70, SEQ ID No71, SEQ ID No72, SEQ ID No73, SEQ ID No74 and SEQ ID No75,
and more particularly the 6 primers SEQ ID No23, SEQ ID No24, SEQ ID No25, SEQ ID No26, SEQ ID No72 and SEQ ID No75.

According to an advantageous embodiment of the method of the invention, the use of the following oligonucleotide primer pairs:
(SEQ ID No23, SEQ ID No24),
(SEQ ID No25, SEQ ID No26),
(SEQ ID No72, SEQ ID No75) and,
permits the obtaining of, respectively:
the fragment SEQ ID No29 of 231 base pairs as defined above,
the fragment SEQ ID No30 of 175 base pairs as defined above,
the fragment SEQ ID No76 of 231 base pairs as defined above.

The primers used in order to effect the amplification by the PCR method of a DNA sequence derived from Struthioniformes, possibly present in a sample of organic material to be analysed, are the 6 primers listed below and as defined above:
SEQ ID No66, SEQ ID No67, SEQ ID No27, SEQ ID No68, SEQ ID No69 and SEQ ID No28, and more particularly the primers SEQ ID No27 and SEQ ID No28.

According to an advantageous embodiment of the method of the invention, the use of the pair of oligonucleotides (SEQ ID No27, SEQ ID No28), permits the obtaining of the fragment SEQ ID No31 of 216 base pairs as defined above.

According to a particularly advantageous mode of implementation of the method of the invention, part of the hybridization steps of the cycles constituting the amplification reaction is carried out at a temperature of 55° C. which is particularly suitable for obtaining a specific amplification. Such a mode of implementation allows a greater specificity of amplification to be obtained.

The visualization of the fragment SEQ ID No29 permits the detection and identification of the presence of chicken (Gallus gallus). The visualization of the fragment SEQ ID No30 permits the detection and identification of the presence of quail (Coturnix coturnix). The visualization of the fragment SEQ ID No76 permits the detection and identification of the presence of turkey (Meleagris gallopavo).

The visualization of the fragment SEQ ID No31 permits the detection and identification of the presence of ostrich (Struthio camelus).

The amplification product obtained, identified using the intermediate strategy or the specific strategy, is generally situated in the 5′ part of the control region for mitochondrial DNA replication, delimited by the nucleotides situated in the vicinity of positions 1 and 439, in particular in the vicinity of positions 80 and 433, and preferably in the vicinity of positions 150 and 433 of the control region for mitochondrial DNA replication, or in the 3′ part of the control region for mitochondrial DNA replication, delimited by the nucleotides situated in the vicinity of positions 750 and 1227, in particular in the vicinity of positions 780 and 1227, and preferably in the vicinity of positions 800 and 1227 of the control region for mitochondrial DNA replication, the said positions being defined according to Desjardins and Morais, 1990, J. Mol. Biol., 599-634.

A subject of the invention is also the use of nucleotide sequences chosen from the oligonucleotide primers as defined above, or the DNA fragments as defined above, for carrying out a method of detecting the presence of biological materials derived from birds, in particular Anseriformes, Galliformes or Struthioniformes, and/or of identifying the species of birds, in particular Anseriformes, Galliformes or Struthioniformes, that is present, in a sample of organic material capable of containing such biological materials.

By way of example of a sample of organic material, there can be cited the agro-food products such as foie gras, pâtés, meats, fats, cooked dishes, eggs etc., manufactured products such as feathers, duvets, costumes with feather accessories, etc. collector's eggs, collector's feathers, bred or wild live animals, stuffed animals etc.

A subject of the invention is also the use as defined above for carrying out a method of detecting the presence of biological materials derived from birds, in particular Anseriformes, Galliformes or Struthioniformes, and/or of identifying the species of birds, in particular Anseriformes, Galliformes or Struthioniformes, that is present in fresh or processed samples of organic materials such as agro-food products, in particular foie gras, pâtés, meats, fats, cooked dishes, eggs, manufactured products such as those based on feathers.

### DESCRIPTION OF THE FIGURES

FIG. 1 represents an agarose gel stained with ethidium bromide. A marker of a size of 100 base pairs (bp) (M) is deposited on the gel. The DNAs are extracted from different samples of organic materials such as feathers (well 1), blood (well 2), bones (well 3), duck mousse (well 4), cooked dishes (well 5), and are deposited on the gel.

FIG. 2 represents an agarose gel stained with ethidium bromide. A marker of a size of 100 bp (M) is deposited on the gel. The DNAs of mallard (well 1), Muscovy duck (well 2), pintail (well 3), eider (well 4), goose (well 5 an (well 6), chicken (well 7), quail (well 8), turkey (well 9) and ostrich (well 10) were amplified using the primers SEQ ID No1 and SEQ ID No5. The amplification product obtained SEQ ID No6 migrates forming a band of 203 base pairs (bp). Well 11 corresponds to a negative amplification control.

FIG. 3 represents alignments of nucleotide sequences of 120 base pairs (120 bp) of the control region for mitochondrial DNA replication of different species of birds, in particular Anseriformes, Galliformes or Struthioniformes. The sequences obtained after sequencing are aligned and compared with the sequences already known. The sequences of the different species of birds represented are respectively the following (from top to bottom in the figure): mallard, pintail, Muscovy duck, tufted duck, mandarin duck, teal, mersander, scoter, barnacle goose, greylag goose, Indian goose, swan, tree duck, chicken, quail and ostrich. The dots represent the bases retained with those of the mallard (reference sequence). The dashes represent deletions of bases.

FIGS. 4-a, 4-b, 4-c, 4-d, 4-e represent the migration profiles expected after enzymatic digestion of the fragment SEQ ID No13 and deposition on a 2% agarose gel after staining with ethidium bromide. A marker of a size of 100 bp (M) is deposited on the gel. FIG. 4-f represents the migration profile obtained after enzymatic digestion of the fragment SEQ ID No13 and deposition on a 2% agarose gel after staining with ethidium bromide. A marker of a size of 100 bp (M) is deposited on the gel. The fragment. SEQ ID No13 is obtained using the primers SEQ ID No11 and SEQ ID No12, by amplification of mtDNA extracted from a cooked dish for which it is desired to detect and identify the genus of bird present in the said dish.

FIG. 4-a represents more particularly the profile expected for the genus Anas. The fragment SEQ ID No13 of 288 bp is deposited in well 1 without having been digested, and therefore serves as a control, comparing it with the other fragments placed in the presence of the different enzymes. Well 2 is the same fragment SEQ ID No13 digested with the enzyme Nsi I, specific to the genus Anas: a cleavage is observed engendering two fragments of 154 and 134 base pairs, which are indeed the fragments specific to the genus Anas. Well 3 is the same fragment SEQ ID No13 digested by the enzyme Acc I, specific to the genus Cairina: no cleavage is observed, given that Acc I is not specific to the genus Anas. Well 4 is the same fragment SEQ ID No13 digested by the enzyme Hha I, specific to the genus Anser: no cleavage is observed. Well 5 is the same fragment SEQ ID No13 digested by the enzyme Nde I, specific to the genus Cygnus: no cleavage is observed. Well 6 is the same fragment SEQ ID No13 digested by the enzyme BsaJ I, specific to the genus Gallus: no cleavage is observed.

The fragment SEQ ID No13 obtained after amplification is each time placed in the presence of all the enzymes, as it may be that there is a mixture of genera, and a profile may be obtained where the enzyme specific to the genus Anas cleaves SEQ ID No13, and the enzyme specific- to the genus Cairina also cleaves SEQ ID No13. The two genera are thus present, hence there is a mixture of species in the sample of organic material.

FIG. 4-b is the profile expected for the genus Cairina. The fragment SEQ ID No13 of 288 bp is deposited in well 1 without having been digested (control). Well 2 is the fragment SEQ ID No13 digested with the enzyme Nsi I: no cleavage is observed, given that Nsi I is not specific to the genus Cairina. Well 3 is the fragment SEQ ID No13 digested by the enzyme Acc I: a cleavage is observed engendering two fragments of 147 and 141 base pairs, which are indeed the fragments specific to the genus Cairina. Well 4 is the fragment SEQ ID No13 digested by the enzyme Hia I: no cleavage is observed. Well 5 is the fragment SEQ ID No13 digested by the enzyme Nde I: no cleavage is observed. Well 6 is the fragment SEQ ID No13 digested by the enzyme BsaJ I: no cleavage is observed.

FIG. 4-c is the profile expected for the genus Anser. The fragment SEQ ID No13 of 408 bp is deposited in well 1 without having been digested (control). Wells 2 and 3 respectively represent the fragment SEQ ID No13 digested with the enzymes Nsi I and Acc I: no cleavage is observed. Well 4 represents the fragment SEQ ID No13 digested with Hia I: a cleavage is observed engendering three fragments of 11, 113 and 284 base pairs, which are indeed the fragments specific to the genus Anser. Wells 5 and 6 respectively represent the fragment SEQ ID No13 digested by the enzyme Nde I and BsaJ I: no cleavage is observed.

FIG. 4-d is the profile expected for the genus Cygnus. The fragment SEQ ID No13 comprising 328 bp is deposited in well 1 without having been digested (control). Wells 2, 3 and 4 respectively represent the fragment SEQ ID No13 digested with the enzymes Nsi I, Acc I and Hha I: no cleavage is observed. Well 5 represents the fragment SEQ ID No13 digested with Nde I: a cleavage is observed engendering two fragments of 132 and 196 base pairs, which are indeed the fragments specific to the genus Cygnus. Well 6 represents the fragment SEQ ID No13 digested by the enzyme BsaJ I: no cleavage is observed.

FIG. 4-e is the profile expected for the genus Gallus. The fragment SEQ ID) No13 of 331 bp is deposited in the well 1 without having been digested (control). Wells 2, 3, 4 and 5 respectively represent the fragment SEQ ID No13 digested with the enzymes Nsi I, Acc I, Hha I and Nde I: no cleavage is observed. Well 6 represents the fragment SEQ ID No13 digested with BsaJ I: a cleavage is observed engendering two fragments of 50 and 281 base pairs, which are indeed the fragments specific to the genus Gallus.

In FIG. 4-f, the example chosen is that of a cooked dish from which the DNA has been extracted and amplified using the primers SEQ ID No11 and SEQ ID No12. Well 1 corresponds to the non-digested amplification fragment SEQ ID No13, of a size of 288 bp (control). The profile obtained in FIG. 4-f, after amplification and digestion the profile 4-f is obtained, is compared with the different profiles expected: it will be seen that the profile 4-f corresponds to that of FIG. 4-a. Thus, the analysed cooked dish does contain a bird of the genus Anas, belonging to the family of the Anatidae.

FIG. 5 represents an agarose gel stained with ethidium bromide on which is deposited a marker of a size of 100 bp (M).

The DNAs of mallard (well 1), Muscovy duck (well 2), goose (well 3) and chicken (well 4) were amplified using the primers SEQ ID No14 and SEQ ID No15, which allow a DNA fragment SEQ ID No20 of a size of 180 bp to be obtained. Only an amplification fragment for the mallard is observed.

The DNAs of mallard (well 5), Muscovy duck (well 6), goose (well 7) and chicken (well 8) were amplified using the primers SEQ ID No16 and SEQ ID No17, which allow a DNA fragment SEQ ID No21 of a size of 191 bp to be obtained. Only an amplification fragment for the Muscovy duck is observed.

The DNAs of mallard (well 9), Muscovy duck (well 10), goose (well 11) and chicken (well 12) were amplified using the primers SEQ ID No18 and SEQ ID No19, which allow a DNA fragment SEQ ID No22 of a size of 176 bp to be obtained. Only an amplification fragment for the greylag goose is observed.

Well 13 represents a negative amplification control.

FIG. 6 represents the positions on the control region for mitochondrial DNA replication, the said positions being defined by Desjardins and Morais, 1990, J. Mol. Biol., 599-634:
of the oligonucleotide primers of the invention, namely SEQ ID No1, SEQ ID No2, SEQ ID No3, SEQ ID No4, SEQ ID No5, SEQ ID No11, SEQ ID No12, SEQ ID No14, SEQ ID No15, SEQ ID No16, SEQ ID No17, SEQ ID No18, SEQ ID No19, SEQ ID No23, SEQ ID No24, SEQ ID No25, SEQ ID No26, SEQ ID No72, SEQ ID No75, SEQ ID No 27, SEQ ID No 28 and,
of the DNA fragments amplified using the primers of the invention, namely SEQ ID No6, SEQ ID No7, SEQ ID No8, SEQ ID No9, SEQ ID No10, SEQ ID No13, SEQ ID No20, SEQ ID No21, SEQ ID No22, SEQ ID No29, SEQ ID No30, SEQ ID No76 and SEQ ID No31.

Table 1 hereafter represents the list of all the existing Anatidae that can be detected and identified using the method of the invention (global strategy).

| LATIN NAME | COMMON NAME |
|---|---|
| Aix galericulata | Mandarin Duck |
| Aix sponsa | Wood Duck |
| Alopochen oegyptiacus | Egyptian Goose |
| Amazonetta brasiliensis | Brazilian Duck |
| Anas acuta | Northern Pintail |
| Anas americana | American Wigeon |
| Anas aucklandica | Brown Teal |
| Anas bahamensis | White-cheeked Pintail |
| Anas bernieri | Madagascar Teal |
| Anas capensis | Cape Teal |
| Anas castenea | Chestnut Teal |
| Anas clypeata | Northern Shoveler |
| Anas crecca | Eurasian Teal |
| Anas cyanoptera | Cinnamon Teal |
| Anas discors | Blue-winged Teal |
| Anas erythrorhyncha | Red-billed Teal |
| Anas falcata | Falcated Duck |
| Anas flavirostris | Speckled Teal |
| Anas formosa | Baikal Teal |
| Anas georgica | Yellow-billed Pintail |
| Anas gibberifrons | Sunda Teal |
| Anas hottenta | Hottentot Teal |
| Anas laysanensis | Laysan Duck |
| Anas luzonica | Philippine Duck |
| Anas melleri | Meller's Duck |
| Anas penelope | Eurasian Wigeon |
| Anas platalea | Red Shoveler |
| Anas plathyrhynchos | Mallard |
| Anas poecilorhyncha | Spotbill Duck |
| Anas querquedula | Garganey |
| Anas rhynchotis | Australian Shoveler |
| Anas rubripes | American Black Duck |
| Anas sibilatrix | Chiloe Wigeon |
| Anas smithi | Cape Shoveler |
| Anas sparsa | African Black Duck |
| Anas specularis | Spectacled Duck |
| Anas strepera | Gadwall |
| Anas superciliosa | Pacific Black Duck |
| Anas undulata | Yellow-billed Duck |
| Anas versicolor | Silver Teal |
| Anas waigiuensis | Salvadori's Teal |
| Anas wywilliana | Hawaiian Duck |
| Anseranas semipalmata | Magpie Goose |
| Anser albifrons | White-fronted Goose |
| Anser anser | Greylag Goose |
| Anser brachyrhynchus | Pink-footed Goose |
| Anser caerulescens | Snow Goose |
| Anser cygnoïdes | Swan Goose |
| Anser erythropus | Lesser White-Fronted Goose |
| Anser fabalis | Bean Goose |
| Anser indicus | Bar-headed Goose |
| Anser rosii | Ross' Goose |
| Anser canagicus | Emperor Goose |
| Anseranas semipalmata | Magpie Goose |
| Aythya affinis | Lesser Scaup |
| Aythya americana | Redhead |
| Aythya australis | Hardhead |
| Aythya baeri | Baer's Pochard |
| Aythya collaris | Ring-necked Duck |
| Aythya fuligula | Tufted Duck |
| Aythya innotata | Madagascar Pochard |
| Aythya marila | Greater Scaup |
| Aythya novoe-seelandioe | New Zealand Scaup |
| Aythya nycora | Ferruginous Duck |
| Aythya vasilineria | Canvasback |
| Biziura lobata | Musk Duck |
| Branta bernicla | Brent Goose |
| Branta canadensis | Canada Goose |
| Branta leucopsis | Barnacle Goose |
| Branta ruficollis | Red-breasted Goose |
| Branta sandvicensis | Hawaiian Goose |
| Bucephala albeola | Bufflehead |
| Bucephala clangula | Goldeneye |
| Bucephala islandica | Barrow's Goldeneye |
| Cairina moschata | Muscovy Duck |
| Cairina scutulata | White-Winged Duck |
| Calonetta leucophrys | Ringed Teal |
| Cereopse novoe-hallandioe | Cape Barren Goose |
| Chauna torquota | Crested Screamer |
| Chenonetta jubata | Australian Wood Duck |
| Chloëphaga hybrida | Kelp Goose |
| Chloëphaga melanoptera | Andean Goose |
| Chloëphaga picta | Magellan Goose |
| Chloëphaga poliocephala | Grey-headed Goose |
| Chloëphaga rubidiceps | Red-headed Goose |
| Clangula hyemalis | Long-tailed Duck |
| Coscoroba coscoroba | Coscoroba Swan |
| Cyanochen cyanopterous | Blue-winged Goose |
| Cygnus atratus | Black Swan |
| Cygnus columbianus | Bewick's Swan |
| Cygnus cygnus | Whooper Swan |
| Cygnus melanocoryphus | Black-necked Swan |
| Cygnus olor | Mute Swan |
| Dendrocygna arborea | West Indian Whistling Duck |
| Dendrocygna arcuata | Wandering Whistling Duck |
| Dendrocycgna autumnalis | Black-bellied Whistling Duck |
| Dendrocycgna bicolor | Fulvous Whistling Duck |
| Dendrocygna eytoni | Plumed Whistling Duck |
| Dendrocycgna guttata | Spotted Whistling Duck |
| Dendrocygna javanica | Lesser Whistling Duck |
| Dendrocycgna viduata | White-faced Whistling Duck |
| Erismature vittata | Argentine blue-billed Duck |
| Heteronetta atricapilla | Black-headed Duck |
| Histrionicus histrionicus | Harlequin Duck |
| Hymenolaimus malacorhynchus | New Zealand Blue Duck |
| Lophonetta specularoides | Spectacled Duck |
| Malacorhynchus mebranaceus | Pink-eared Duck |
| Marmaronetta angustirostris | Marbled Duck |
| Melanitta fusca | Velvet Scoter |
| Melanitta nigra | Common Scoter |
| Melanitta perspicillata | Surf Scoter |
| Merganetta armata | Torrent Duck |
| Mergus albellus | Smew |
| Mergus cucullatus | Hooded Merganser |
| Mergus merganser | Goosander |
| Mergus octosetaceus | Brazilian Merganser |
| Mergus serrator | Red-breasted Merganser |
| Mergus squamatus | Chinese Merganser |
| Neochen jubatus | Orinoco Goose |
| Netta erythrophthalma | Southern Pochard |
| Netta peposaca | Rosy-billed Pochard |
| Netta rufina | Red-crested Pochard |
| Nettapus auritus | African Pygmy Goose |
| Nettapus coromandelianus | Cotton Pygmy Goose |
| Nettapus pulchellus | Green Pygmy Goose |
| Oxyura australis | Australian blue-billed Duck |
| Oxyura dominica | Masked Duck |
| Oxyura jamaicensis | Ruddy Duck |
| Oxyura leucocephala | White-headed Duck |
| Oxyura maccoa | Maccoa Duck |
| Oxyura vittata | Lake Duck |
| Plectropterous gambensis | Spur-Winged Goose |
| Polysticta stelleri | Steller's Eider |
| Pteronetta hartlaubi | Hartlaub's Duck |
| Sarkidiornis melanotos | Comb Duck |
| Somateria fischeri | Spectacled Eider |
| Somateria mollissima | Common Eider |
| Somateria spectabilis | King Eider |
| Stictonetta noevosa | Freckled Duck |
| Tachyeres brachypterus | Steamerduck |
| Tachyeres patachonicus | Flying Steamerduck |
| Tachyeres pteneres | Flightless Steamerduck |
| Tadorna cana | South African Shelduck |
| Tadorna ferruginea | Ruddy Shelduck |
| Tadorna tadorna | Common Shelduck |
| Tadorna tadornoides | Australian Shelduck |
| Tadorna radjah | Radjah Shelduck |
| Tadorna variegata | Paradise Shelduck |
| Thalassornis leuconotus | White-backed Duck |

The examples hereafter illustrate the invention. They in no way limit it.

### EXAMPLE 1

### Extraction of DNA

In order to be able to develop a method for the detection by gene amplification of biological materials derived from Anatidae in products used in agro-food production and in all the other fields using organic material, the experiments described in this first example were realized with various types of sources in which biological materials derived from Anatidae are potentially present.

1) Extraction of DNA by the Phenol/Chloroform Method

This method is used for all the types of samples likely to contain organic material, such as feathers, fresh tissues, processed products, blood, fats, bones.

This method employs techniques described in the references HÄNNI et al., 1990, C. R. Acad. Sci. Paris., 310, 365-370 and HÄNNI et al., 1995, Nucl. Acids Res., 23, 881-882, concerning the extraction of DNA from bones and teeth.

A quantity of approximately 1 to 2 g of sample of organic material is incubated for two hours at 37° C. in 40011 of lysis buffer of the following composition:
STE 1× (NaCl 10 mM, Tris 10 mM at pH 7.4, EDTA (ethylenediaminetetraacetic acid) 1 mM),
22% SDS,
proteinase K at 0.5 mg/ml.

The proteinase K permits the degradation of the proteins and the release of the nucleic acids. The lysate is then extracted twice by a volume of phenol/chloroform (1/1). The product is centrifuged for 15 minutes at 1000 g, the organic phase is eliminated, which allows the proteic part of the lysate to be discarded. The DNA is precipitated by 1/10 volume of 2M sodium acetate then by 2.5 volumes of isopropanol and centrifuged for 30 minutes at 10 000 g. The DNA is taken up in water: a DNA extract is thus obtained. The volume of water is a function of the quantity of DNA recovered.

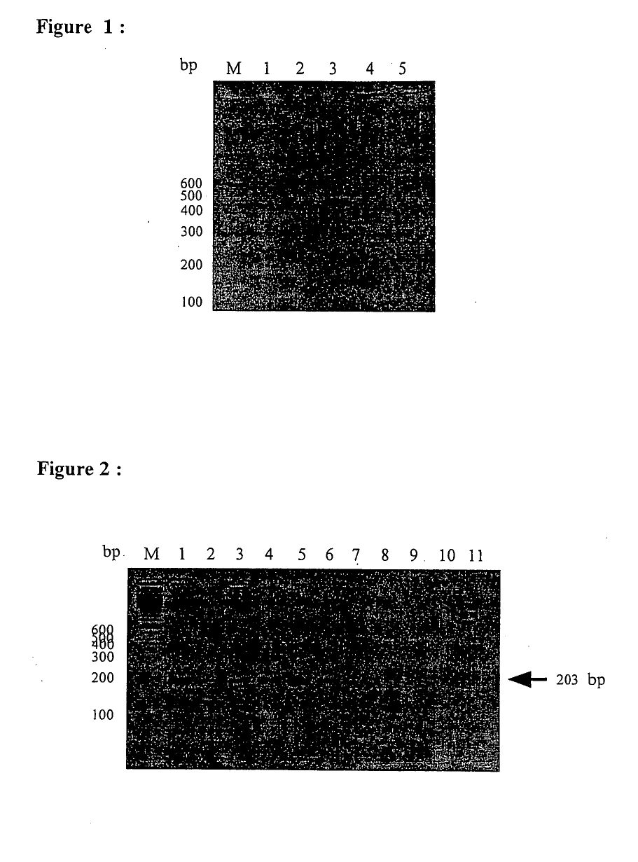

The migration of the DNAs derived from the various samples gives streaks characteristic of a degraded DNA, which is however wholly amplifiable (FIG. 1).

2) Extraction of DNA by the Extraction Kit Method

This method is realized in, the conditions described by the supplier Qiagen.

### EXAMPLE 2

### Amplification of DNA

The PCR is carried out with the pair of primers SEQ ID No1 and SEQ ID No5 defined above. The amplifications are realized in a total volume of 50 μl containing:
200 μg/ml of BSA (Bovine Serum Albumin),
250 mM of dNTP (desoxynucleotide Triphosphate),
300 ng of each primer,
1.5 mM of magnesium chloride (MgCl₂),
PCR 10× buffer (100 mM Tris-HCl pH 8.3; 500 mM KCl),
1 unit of Taq Polymerase,
qsf 50 μl of sterile distilled water,
1 μl of DNA extract.

The reaction mixture is carried out in a sterile room under a horizontal-flow hood, to avoid contaminations as much as possible. The PCRs are carried out on a PCR Ependorff device. Each PCR is broken down as follows:
1 initial cycle at a temperature of 94° C. for 2 minutes then,
40 cycles at a temperature of 94° C. for 1 minute, at a temperature of 55° C. to 63° C. for 1 minute, at a temperature of 72° C. for 2 minutes; with the last cycle a terminal elongation is effected at a temperature of 72° C. for 7 minutes.

The amplification products are analysed by 2% agarose gel electrophoresis under a constant voltage of 100 V for 30 min, and using ethidium bromide for the visualization of the amplifications obtained.

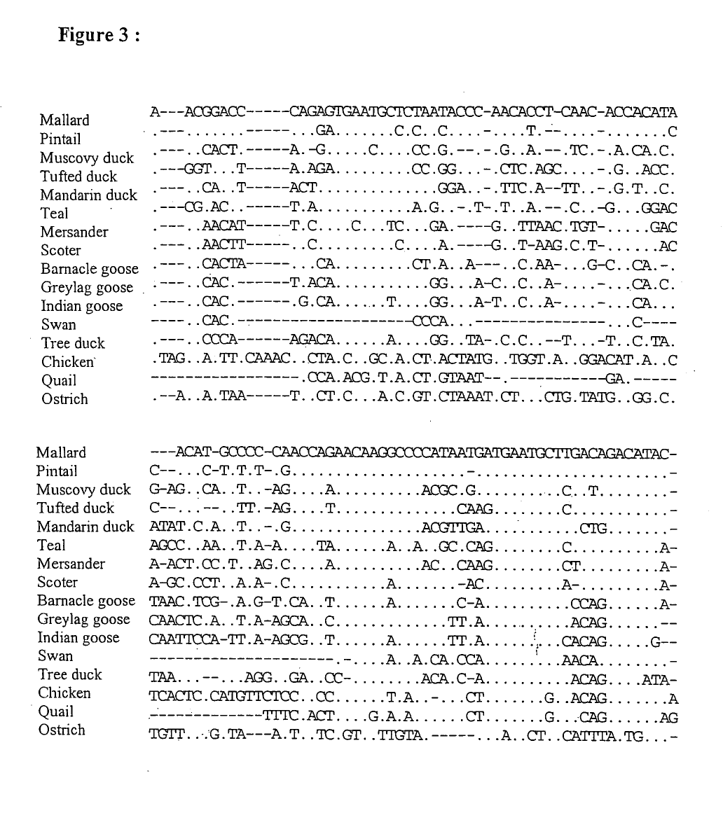

The PCR reaction using the pair of primers SEQ ID No1 and SEQ ID No5 was carried out on a series of DNA extracts from birds. A single amplification product is observed (fragment SEQ ID No6 of a length of 203 base pairs) for the DNAs of Anseriformes, and in particular of Anatidae. By contrast, no amplification band is observed for chicken (well 7), quail (well 8), turkey (well 9) and ostrich (well 10): thus, the primers SEQ ID No1 and SEQ ID No5 are quite specific to the Anseriformes (FIG. 2).

The whole of the PCR product can be purified directly with the “QIAquick PCR Purification Kit” system from Qiagen under the conditions stated. The whole of the PCR product is passed over a column of silica gel. The nucleic acids are retained on the column while the other residues of the PCR are eluted using microcentrifugation. The impurities are eliminated and the DNA is eluted by Tris buffer or water. The thus-purified DNA is visualized and quantified on 1% agarose gel.

### EXAMPLE 3

### Characterization of the Fragments by Direct Sequencing (“Global Strategy”)

The automatic sequencing is carried out on the purified PCR products.

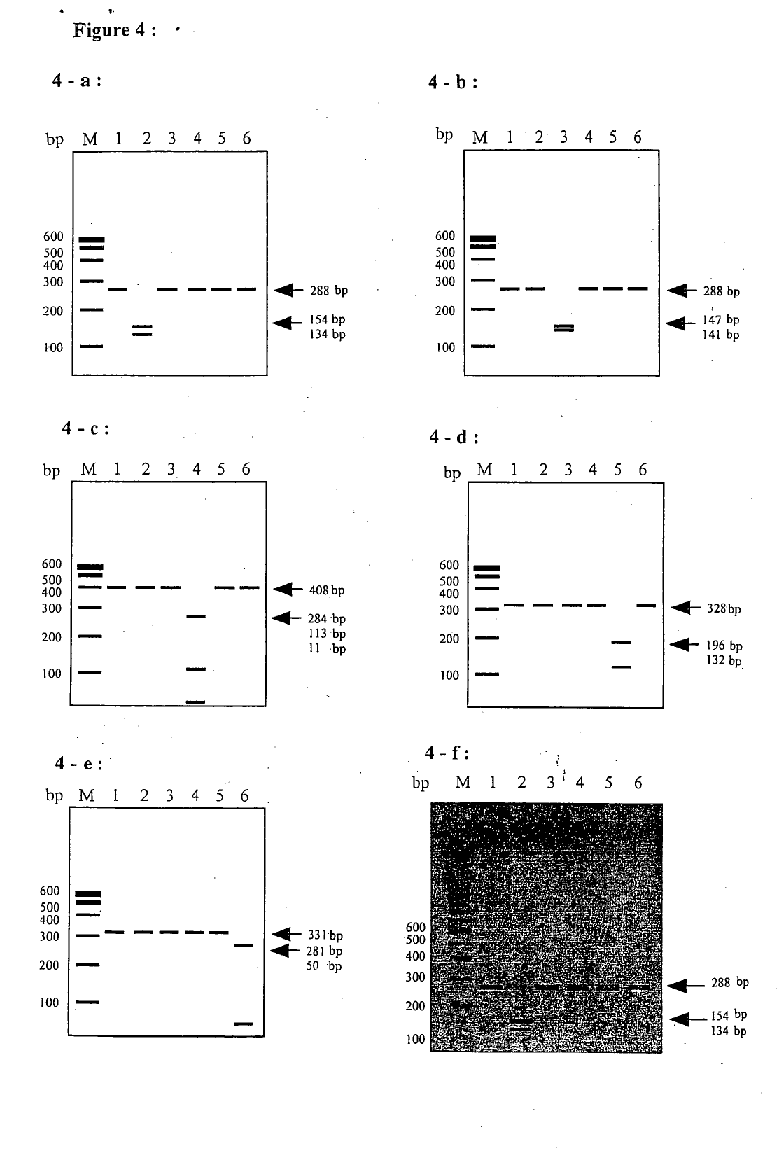

The primers used are those which served to amplify the fragment in question. The “Perkin-Elmer” kit is used for the PCR reaction, which is carried out over 25 cycles in the conditions stated by the supplier. The amplification products obtained at the end of the PCR reaction are precipitated with ethanol and deposited on a polyacrylamide gel. The sequences obtained are then compared with those of the banks or with the sequences already obtained (FIG. 3).

### EXAMPLE 4

### Characterization of the Fragments Extracted by Enzymatic Digestion (“Intermediate Strategy”)

A restriction map is constructed for each of the genera most frequently used in the composition of organic substances based on Anatidae and Gallinaceans. The genera chosen as references are the following:
the genus Anas for the mallard (Anas platyrhynchos), the pintail (Anas acuta) or the garganey (Anas querquedula),
the genus Cairina for the Muscovy duck (Cairina moschata),
the genus Anser for the greylag goose (Anser anser), the snow goose (Anser caerulescens),
the genus Cygnus for the whooper swan (Cygnus Cygnus), the black swan (Cygnus atratus),
the genus Gallus for the chicken (Gallus gallus).

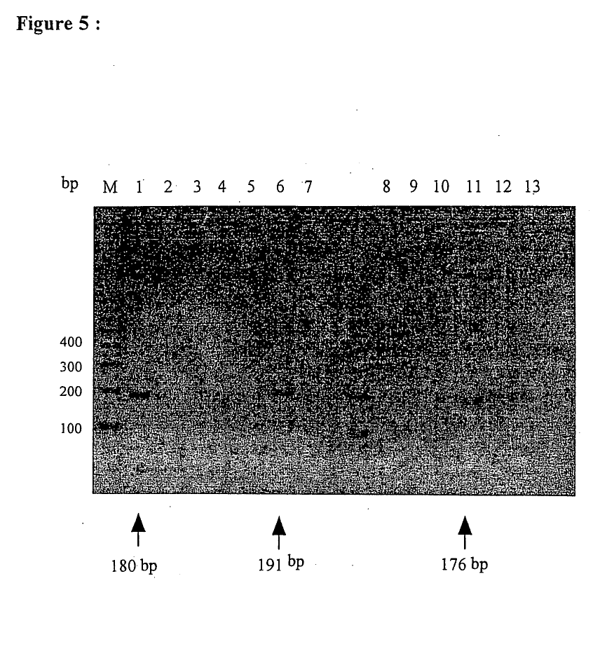

The example chosen is that of a cooked dish for which it is wished to detect and identify the presence of biological materials derived from birds (cf FIGS. 4-a to 4-f). The DNA is extracted from the cooked dish and is amplified using the primers SEQ ID No11 and SEQ ID No12.

The enzymes were chosen in order to have cleavage sites that were different and specific to the genera analysed for the fragment SEQ ID No13, obtained using the primers SEQ ID No11 and SEQ ID No12 by amplification. It will be recalled that the size of the fragment SEQ ID No13 varies between the genera of Anatidae or Gallinaceans, because of insertions or deletions of some DNA bases for the duck, goose, swan and chicken.

The specificity of the restriction enzymes used is recalled in table 2 below. A tenth of the PCR reactions were used directly to realize each enzymatic digestion with the buffers specific to the cited enzymes (Sambrook et al., 1987). The digestions were carried out for 30 minutes to 37° C. The digestion products were tested on a 3% agarose gel.

Two fragments of a size of 154 bp and 134 bp are obtained. The analysed cooked dish therefore contains duck of the genus Anas. The same fragment SEQ ID No13 is digested by other restriction enzymes specific to other genera: no cleavage is observed for the other restriction enzymes. The cooked dish is therefore based only on Anas (FIG. 4-a to 4-f).

Table 2 below represents the list of the restriction enzymes used for the fragment SEQ ID No13.

| Anatidae | Restriction | Number of | Size of the bands |
|---|---|---|---|
| Gallinaceans | enzymes used | bands | in base pairs |
| Duck | Nsi I | 2 | 154 |
| (genus Anas) | | | 134 |
| duck | Acc I | 2 | 147 |
| (genus Cairina) | | | 141 |
| goose | Hha I | 3 | 11 |
| (genus Anser) | | | 113 |
| | | | 284 |
| swan | Nde I | 2 | 132 |
| (genus Cygnus) | | | 196 |
| chicken | BsaJ I | 2 | 50 |
| (genus Gallus) | | | 281 |

### EXAMPLE 5

### “Specific Strategy”

The PCR is carried out with the primer pairs (SEQ ID No14, SEQ ID No15), (SEQ ID No16, SEQ ID No17) and (SEQ ID No18, SEQ ID No19).

The amplifications are realized in a total volume of 50 μl containing:
200 μg/ml of BSA,
250 mM of dNTP,
300 ng of each primer,
1.5 mM of MgCl₂,
PCR 10× buffer (100 mM Tris-HCl pH 8.3; 500 mM KCl),
1 unit of Taq Polymerase,
qsf 50 μl of sterile distilled water,
1 μl of DNA extract

The reaction mixture is carried out in a sterile room under a horizontal-flow hood, in order to avoid contaminations as much as possible. The PCRs are carried out on an Ependorff PCR device.

Each PCR is broken down as follows:
1 initial cycle at a temperature of 94° C. for 2 minutes then,
40 cycles at a temperature of 94° C. for 1 minute, at a temperature of 63° C. for 1 minute, at a temperature of 72° C. for 2 minutes; with the last cycle a terminal elongation is effected at a temperature of 72° C. for 7 minutes.

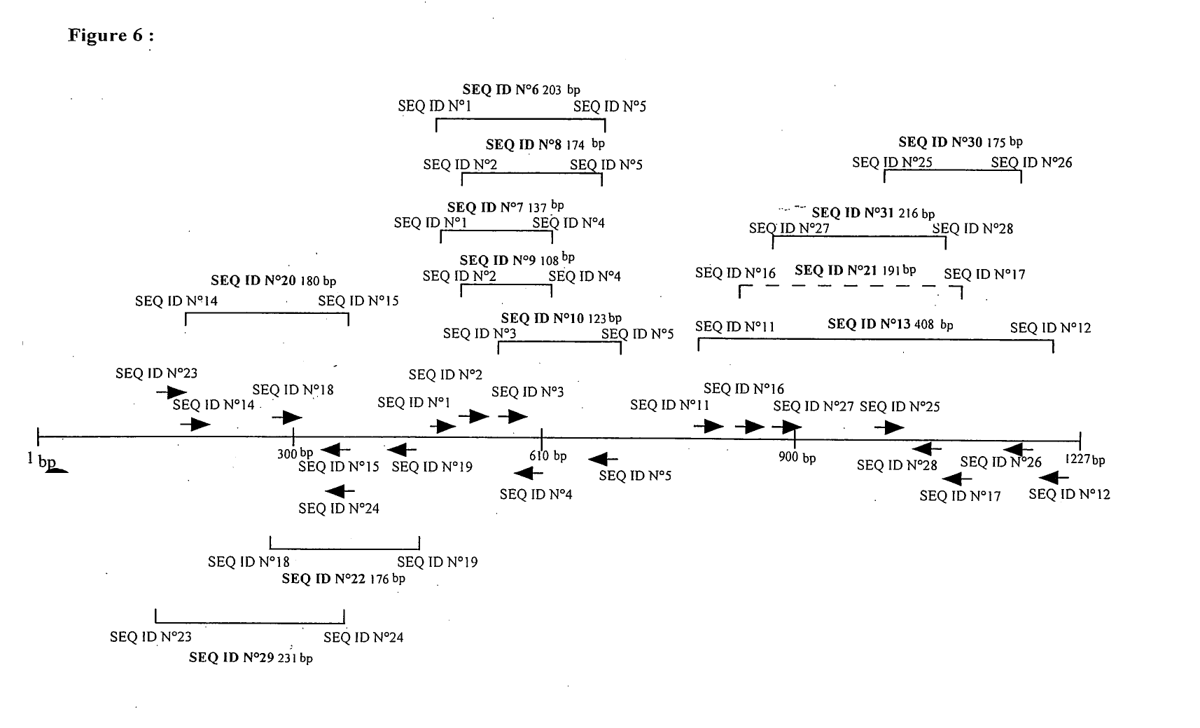

The amplification products are analysed by 2% agarose gel electrophoresis under a constant voltage of 100 V for 30 min, and using ethidium bromide for the visualization of the amplifications obtained (FIG. 5).

The use of the primers SEQ ID No14 and SEQ ID No15 allows an amplification product SEQ ID No20 to be obtained which migrates forming a band of 180 base pairs: this fragment is quite specific to the mallard (well 1) since no other band is observed for the other species. The primers SEQ ID No14 and SEQ ID No15 are therefore quite specific to the mallard. The use of the primers SEQ ID No16 and SEQ ID No17 allows an amplification fragment SEQ ID No21 to be obtained which migrates forming a band of 191 base pairs: this fragment is quite specific to the Muscovy duck (well 6) since no other band is observed for the other species. The primers SEQ ID No16 and SEQ ID No17 are therefore quite specific to the Muscovy duck. The use of the primers SEQ ID No18 and SEQ ED No19 allows an amplification fragment SEQ ID No22 to be obtained which migrates forming a band of 176 base pairs: this fragment is quite specific for the greylag goose (well 11) since no other band is observed for the other species. The primers SEQ ID No18 and SEQ ID No19 are therefore quite specific to the greylag goose.

The chicken is a control (well 13) and makes it possible to demonstrate that the three primer pairs are quite specific to the Anseriformes (FIG. 5).

1. Method of detecting the presence of biological materials derived from birds, in particular from Anseriformes, Galliformes or Struthioniformes, in a sample of organic material, characterized in that the presence of DNA derived from birds, in particular from Anseriformes, Galliformes or Struthioniformes, in the said organic material is determined by amplification of a DNA sequence specific to the genome of birds, in particular of the Anseriformes, Galliformes or Struthioniformes, and contained in the DNA extracted from the said sample, namely a sequence present in the genomes of birds, in particular the Anseriformes, Galliformes or Struthioniformes but absent from the genomes of other animal genera, and in particular of other animal species.

2. Method of detecting the presence of biological materials derived from birds, in particular from the Anseriformes, Galliformes or Struthioniformes, in a sample of organic material and of identifying the genus, in particular the species of bird, in particular Anseriforme, Galliforme or Struthioniforme, present in the said sample, characterized in that the presence of DNA derived from birds, in particular from Anseriformes, Galliformes or Struthioniformes, in the said organic material is determined by amplification of a DNA sequence specific to the genome of birds, and in particular the Anseriformes, Galliformes or Struthioniformes, and contained in the DNA extracted from the said sample, namely a sequence present in the genomes of birds, and in particular the Anseriformes, Galliformes or Struthioniformes, but absent from the genomes of other animal genera, in particular of other animal species, and in that the DNA sequence specific to the genome of birds, in particular of the Anseriformes, Galliformes or Struthioniformes, thus amplified, is compared with other DNA sequences of the genome of birds, in particular of the Anseriformes, Galliformes or Struthioniformes, the said DNA sequence specific to the genome of birds, in particular the Anseriformes, Galliformes or Struthioniformes, thus amplified, presenting at least 50% identity, in particular 60% identity with the other aforementioned DNA sequences of the genome of birds, and in particular of the Anseriformes, Galliformes or Struthioniformes.

3. Method according to claim 1, characterized in that it permits the detection and/or identification of the presence of Anseriformes, in particular of Anatidae chosen from the group constituted by the ducks such as the pintail (Anas acuta), mandarin duck (Aix galericulata), shoveler (Anas clypeata), garganey (Anas querquedula), eider (Somateria mollissima), geese such as the lesser white-fronted goose (Anser erythropus), snow goose (Anser caerulescens), Canada goose (Branta canadensis), and swans such as the whooper swan (Cygnus cygnus), black swan (Cygnus atratus), mute swan (Cygnus olor) and in particular the domestic species of ducks such as the mallard (Anas platyrhynchos), Muscovy duck (Cairina moschata) or the domestic species of goose such as the greylag goose (Anser anser).

4. Method according to claim 1, characterized in that the amplified sequence of the genome of birds, in particular of the Anseriformes, Galliformes or Struthioniformes, is of mitochondrial origin.

5. Method according to claim 1, characterized in that the amplified sequence of the genome of the birds, in particular of the Anseriformes, Galliformes or Struthioniformes, is situated in the central part of the control region for mitochondrial DNA replication, delimited by the nucleotides situated in the vicinity of positions 440 and 750, and in particular in the vicinity of positions 450 and 700, and preferably in the vicinity of positions 459 and 665 of the control region for mitochondrial DNA replication.

6. Method according to any claim 1, characterized in that the amplified sequence of the genome of the birds, in particular of the Anseriformes, Galliformes or Struthioniformes, is situated in the 5′ part of the control region for mitochondrial DNA replication, delimited by the nucleotides situated in the vicinity of positions 1 and 439, and in particular in the vicinity of positions 80 and 433, and preferably in the vicinity of positions 150 and 433 of the control region for mitochondrial DNA replication, or in the 3′ part of the control region for mitochondrial DNA replication, delimited by the nucleotides situated in the vicinity of positions 750 and 1227, and in particular in the vicinity of positions 780 and 1227, and preferably in the vicinity of positions 800 and 1227 of the control region for mitochondrial DNA replication.

7. Oligonucleotides characterized in that they are chosen from those:
(1)—presenting a sequence identity of at least 80%, preferably 90% and advantageously 95% with an oligonucleotide constituted by a sequence of approximately 15 to 25 nucleotides, in particular 20 to 25 nucleotides, contained in the following sequence SEQ ID No32 (positions 455 to 479 according to Desjardins and Morais, 1990, J. Mol. Biol., 212, 599-634):
ACG TGA CYA GCY TCA GGC CCA TAC G
in which Y is C or T,
or comprising the following sequence SEQ ID No33 (positions 465 to 479 according to Desjardins and Morais, 1990, J. Mol. Biol., 212, 599-634):
CTT CAG GCC CAT ACG
or constituted by the following sequence SEQ ID No11 (positions 459 to 479 according to Desjardins and Morais, 1990, J. Mol. Biol., 212, 599-634):
ACT AGC TTC AGG CCC ATA CG
or those,
(2)—presenting a sequence identity of at least 80%, preferably 90% and advantageously 95% with an oligonucleotide constituted by a sequence of approximately 15 to 25 nucleotides, in particular 20 to 25 nucleotides, contained in the following sequence SEQ ID No34 (positions 484 to 508 according to Desjardins and Morais, 1990, J. Mol. Biol., 212, 599-634):
CCC TAA ACC CCT CGC CCT CCT CAC A
or comprising the following sequence SEQ ID No35 (positions 493 to 508 according to Desjardins and Morais, 1990, J. Mol. Biol., 212, 599-634):
CTC GCC CTC CTC ACA
or constituted by the following sequence SEQ ID No2 (positions 488 to 508 according to Desjardins and Morais, 1990, J. Mol. Biol., 212, 599-634):
AAC CCC TCG CCC TCC TCA CA
or those,
(3)—presenting a sequence identity of at least 80%, preferably 90% and advantageously 95% with an oligonucleotide constituted by a sequence of approximately 15 to 25 nucleotides, in particular 20 to 25 nucleotides, contained in the following sequence SEQ ID No36 (positions 537 to 561 according to Desjardins and Morais, 1990, J. Mol. Biol., 212, 599-634):
GGG CCA TCM ATT GGG TTC ACT CAC C
in which M is A or C,
or comprising the following sequence SEQ ID No37 (positions 547 to 561 according to Desjardins and Morais, 1990, J. Mol. Biol., 212, 599-634):
TTG GGT TCA CTC ACC
or constituted by the following sequence SEQ ID No3 (positions 542 to 561 according to Desjardins and Morais, 1990, J. Mol. Biol., 212, 599-634):
ATC AAT TGG GTT CAC TCA CC
or those,
(4)—presenting a sequence identity of at least 80%, preferably 90% and advantageously 95% with an oligonucleotide constituted by a sequence of approximately 15 to 25 nucleotides, in particular 20 to 25 nucleotides, contained in the following sequence SEQ ID No38 (positions 574 to 599 according to Desjardins and Morais, 1990, J. Mol. Biol., 212, 599-634):
GGT RRA VST MNT CCA CAG ATG CCA C
in which R is A or G, V is A, C or G, N is A, C, G or T, S is C or G, M is A or C,
or comprising the following sequence SEQ ID No39 (positions 584 to 599 according to Desjardins and Morais, 1990, J. Mol. Biol., 212, 599-634):
TTC CAC AGA TGC CAC
or constituted by the following sequence SEQ ID No4 (positions 580 to 599 according to Desjardins and Morais, 1990, J. Mol. Biol., 212, 599-634):
AAG TAT TCC ACA GAT GCC AC
or those,
(5)—presenting a sequence identity of at least 80%, preferably 90% and advantageously 95% with an oligonucleotide constituted by a sequence of approximately 15 to 25 nucleotides, in particular 20 to 25 nucleotides, contained in the following sequence SEQ ID No40 (positions 639 to 664 according to Desjardins and Morais, 1990, J. Mol. Biol., 212, 599-634):
CMG GAA RAR ADA ARW RGA ACC AGA G
in which R is A or G, D is A, G or T, M is A or C, W is A or T,
or comprising the following sequence SEQ ID No41 (positions 648 to 664 according to Desjardins and Morais, 1990, J. Mol. Biol., 212, 599-634):
A TAA AAA GAA CCA GAG
or constituted by the following sequence SEQ ID No5 (positions 645 to 664 according to Desjardins and Morais, 1990, J. Mol. Biol., 212, 599-634):
AAA AAT AAA AGG AAC CAG AG
or those,
(6)—presenting a sequence identity of at least 80%, preferably 90% and advantageously 95% with an oligonucleotide constituted by a sequence of approximately 15 to 25 nucleotides, in particular 20 to 25 nucleotides, contained in the following sequence SEQ ID No42 (positions 797 to 820 according to Desjardins and Morais, 1990, J. Mol. Biol., 212, 599-634):
WVK CAY MYY KRY ACT GAT GCA CTT T
in which Y is C or T, K is G or T, R is A or G, M is A or C, W is A or T, V is A, C or G, M is A or C,
or comprising the following sequence SEQ ID No43 (positions 807 to 820 according to Desjardins and Morais, 1990, J. Mol. Biol., 212, 599-634):
ACA CTG ATG CAC TTT
or constituted by the following sequence SEQ ID No11 (positions 802 to 820 according to Desjardins and Morais, 1990, J. Mol. Biol., 212, 599-634):
CCT TGA CAC TGA TGC ACT TT
or those,
(7)—presenting a sequence identity of at least 80%, preferably 90% and advantageously 95% with an oligonucleotide constituted by a sequence of approximately 15 to 25 nucleotides, in particular 20 to 25 nucleotides, contained in the following sequence SEQ ID No44 (positions 1220 to 1224 according to Desjardins and Morais, 1990, J. Mol. Biol., 212, 599-634):
GCY TTD DND KKR WRS WAT GTG GAC G
in which Y is C or T, D is A, G or T, B is C, G or T, K is G or T, W is A or T, N is A, C, G or T, R is A or G, and S is C or G,
or comprising the following sequence SEQ ID No45 (positions 1210 to 1224 according to Desjardins and Morais, 1990, J. Mol. Biol., 212, 599-634):
TAA GCT ATG TGG ACG
or constituted by the following sequence SEQ ID No12 (positions 1205 to 1224 according to Desjardins and Morais, 1990, J. Mol. Biol., 212, 599-634):
TGT GGT AAG CTA TGT GGA CG
or those,
(8)—presenting a sequence identity of at least 80%, preferably 90% and advantageously 95% with an oligonucleotide constituted by a sequence of approximately 15 to 25 nucleotides, in particular 20 to 25 nucleotides, contained in the following sequence SEQ ID No46 (positions 180 to 206 according to Desjardins and Morais, 1990, J. Mol. Biol., 212, 599-634):
MTN MHM BDN HMV HVN NCA TAC CCT A
in which Y is C or T, M is A or C, H is A, C or T, B is C, G or T, D is A, G or T, N is A, C, G or T, W is A or T, V is A, C or G,
or comprising the following sequence SEQ ID No47 (positions 190 to 206 according to Desjardins and Morais, 1990, J. Mol. Biol., 212, 599-634):
AAC GGA CAT ACC CTA
or constituted by the following sequence SEQ ID No14 (positions 185 to 206 according to Desjardins and Morais, 1990, J. Mol. Biol., 212, 599-634):
CAT GTC AAC GGA CAT ACC CTA
or those,
(9)—presenting a sequence identity of at least 80%, preferably 90% and advantageously 95% with an oligonucleotide constituted by a sequence of approximately 15 to 25 nucleotides, in particular 20 to 25 nucleotides, contained in the following sequence SEQ ID No48 (positions 355 to 376 according to Desjardins and Morais, 1990, J. Mol. Biol., 212, 599-634):
CAT RAG NWA WKG KYG GGT GGA GAG G
in which R is A or G, N is A, C, G or T, W is A or T, K is G or T, Y is C or T,
or comprising the following sequence SEQ ID No49 (positions 365 to 376 according to Desjardins and Morais, 1990, J. Mol. Biol., 212, 599-634):
GGG TGG GTG GAG AGG
or constituted by the following sequence SEQ ID No15 (positions 361 to 376 according to Desjardins and Morais, 1990, J. Mol. Biol., 212, 599-634):
TAA TGG GTG GGT GGA GAG G
or those,
(10)—presenting a sequence identity of at least 80%, preferably 90% and advantageously 95% with an oligonucleotide constituted by a sequence of approximately 15 to 25 nucleotides, in particular 20 to 25 nucleotides, contained in the following sequence SEQ ID No50 (positions 833 to 855 according to Desjardins and Morais, 1990, J. Mol. Biol., 212, 599-634):
GTT AWK GYY MYN CTC CGC RGC CCT T
in which W is A or T, K is G or T, Y is C or T, M is A or C, Y is C or T, N is A, C, G or T, R is A or G,
or comprising the following sequence SEQ ID No51 (positions 843 to 855 according to Desjardins and Morais, 1990, J. Mol. Biol., 212, 599-634):
TCC TCC GCA GCC CTT
or constituted by the following sequence SEQ ID No16 (positions 837 to 855 according to Desjardins and Morais, 1990, J. Mol. Biol., 212, 599-634):
ATG CTC TCC TCC GCA GCC CTT
or those,
(11)—presenting a sequence identity of at least 80%, preferably 90% and advantageously 95% with an oligonucleotide constituted by a sequence of approximately 15 to 25 nucleotides, in particular 20 to 25 nucleotides, contained in the following sequence SEQ ID No52 (positions 1120 to 1150 according to Desjardins and Morais, 1990, J. Mol. Biol., 212, 599-634):
DTW ATG ATT HTY RTT ADA TTA CGC T
in which D is A, G or T, W is A or T, H is A, C or T, Y is C or T, R is A or G,
or comprising the following sequence SEQ ID No53 (positions 1130 to 1150 according to Desjardins and Morais, 1990, J. Mol. Biol., 212, 599-634):
TCG TTA GAT TAC GCT
or constituted by the following sequence SEQ ID No17 (positions 1125 to 1150 according to Desjardins and Morais, 1990, J. Mol. Biol., 212, 599-634):
GAT TGT CGT TAG ATT ACG CT
or those,
(12)—presenting a sequence identity of at least 80%, preferably 90% and advantageously 95% with an oligonucleotide constituted by a sequence of approximately 15 to 25 nucleotides, in particular 20 to 25 nucleotides, contained in the following sequence SEQ ID No54 (positions 249 to 272 according to Desjardins and Morais, 1990, J. Mol. Biol., 212, 599-634):
AAY GHA TGA AYG HYC AGN DAC CAT
in which Y is C or T, H is A, C or T, N is A, C, G or T, D is A, G or T,
or comprising the following sequence SEQ ID No55 (positions 256 to 272 according to Desjardins and Morais, 1990, J. Mol. Biol., 212, 599-634):
ATG TTC TAG GAC CAT
or constituted by the following sequence SEQ ID No18 (positions 252 to 272 according to Desjardins and Morais, 1990, J. Mol. Biol., 212, 599-634):
ATG AAT GTT CTA GGA CCA T
or those,
(13)—presenting a sequence identity of at least 80%, preferably 90% and advantageously 95% with an oligonucleotide constituted by a sequence of approximately 15 to 25 nucleotides, in particular 20 to 25 nucleotides, contained in the following sequence SEQ ID No56 (positions 409 to 433 according to Desjardins and Morais, 1990, J. Mol. Biol., 212, 599-634):
CGG GTT CTG ATT TCA CGT GAG GAG T
or comprising the following sequence SEQ ID No57 (positions 419 to 433 according to Desjardins and Morais, 1990, J. Mol. Biol., 212, 599-634):
TTT CAC GTG AGG AGT
or constituted by the following sequence SEQ ID No19 (positions 414 to 433 according to Desjardins and Morais, 1990, J. Mol. Biol., 212, 599-634):
TCT GAT TTC ACG TGA GGA GT.

8. Primer pairs characterized in that they are constituted:
by any one of the oligonucleotides SEQ ID No1, SEQ ID No32, SEQ ID No33, SEQ ID No2, SEQ ID No34, SEQ ID No35, SEQ ID No3, SEQ ID No36, SEQ ID No37 and any one of the oligonucleotides SEQ ID No4, SEQ ID No38, SEQ ID No39, SEQ ID No5, SEQ ID No40, SEQ ID No41 according to claim 7 or,
by any one of the oligonucleotides SEQ ID No11, SEQ ID No42, SEQ ID No43 and any one of the oligonucleotides SEQ ID No12, SEQ ID No44, SEQ ID No45 according to claim 7 or,
by any one of the oligonucleotides SEQ ID No14, SEQ ID No46, SEQ ID No47, and any one of the oligonucleotides SEQ ID No15, SEQ ID No48, SEQ ID No49 according to claim 7 or,
by any one of the oligonucleotides SEQ ID No16, SEQ ID No50, SEQ ID No51, and any one of the oligonucleotides SEQ ID No17, SEQ ID No52, SEQ ID No53 according to claim 7 or,
by any one of the oligonucleotides SEQ ID No18, SEQ ID No54, SEQ ID No55, and any one of the oligonucleotides SEQ ID No19, SEQ ID No56, SEQ ID No57 according to claim 7,
and advantageously constituted by the pair of olignucleotides chosen from the following pairs:
SEQ ID No 1 and SEQ ID No 5,
SEQ ID No 2 and SEQ ID No 5,
SEQ ID No 1 and SEQ ID No 4,
SEQ ID No 2 and SEQ ID No 4,
SEQ ID No 3 and SEQ ID No 5,
SEQ ID No 11 and SEQ ID No 12,
SEQ ID No 14 and SEQ ID No 15,
SEQ ID No 16 and SEQ ID No 17,
SEQ ID No 18 and SEQ ID No 19.

9. Olignucleotides characterized in that they are chosen from those:
(1)—presenting a sequence identity of at least 80%, preferably 90% and advantageously 95% with an oligonucleotide constituted by a sequence of approximately 15 to 25 nucleotides, in particular 20 to 25 nucleotides, contained in the following sequence SEQ ID No58 (positions 145 to 170 according to Desjardins and Morais, 1990, J. Mol. Biol., 212, 599-623):
ATA TGK MVN CGG CAT TAA CCT ATA T
in which K is G or T, M is A or C, V is A, C or G, N is A, C, G or T,
or comprising the following sequence SEQ ID No59 (positions 155 to 170 according to Desjardins and Morais, 1990, J. Mol. Biol., 212, 599-634):
GGC ATT AAC CTA TAT
or constituted by the following sequence SEQ ID No23 (positions 151 to 170 according to Desjardins and Morais, 1990, J. Mol. Biol., 212, 599-634):
ATA CGG GCA TTA ACC TAT AT
or those,
(2)—presenting a sequence identity of at least 80%, preferably 90% and advantagelously 95% with an oligonucleotide constituted by a sequence of approximately 15 to 25 nucleotides, in particular 20 to 25 nucleotides, contained in the following sequence SEQ ID No60 (positions 356 to 380 according to Desjardins and Morais, 1990, J. Mol. Biol., 212, 599-634):
GCG AGY ATA ACC AAA TSG GTT ACA T
in which Y is C or T, S is C or G,
or comprising the following sequence SEQ ID No61 (positions 366 to 380 according to Desjardins and Morais, 1990, J. Mol. Biol., 212, 599-634):
CCA AAT GGG TTA CAT
or constituted by the following sequence SEQ ID No24 (positions 361 to 380 according to Desjardins and Morais, 1990, J. Mol. Biol., 212, 599-634):
CAT AAC CAA ATG GGT TAC AT
or those,
(3)—presenting a sequence identity of at least 80%, preferably 90% and advantageously 95% with an oligonucleotide constituted by a sequence of approximately 15 to 25 nucleotides, in particular 20 to 25 nucleotides, contained in the following sequence SEQ ID No62 (positions 1014 to 1037 according to Desjardins and Morais, 1990, J. Mol. Biol., 212, 599-634):
TAC ATA CAW ACY MWC CGC ACA AAT A
in which W is A or T, Y is C or T, M is A or C,
or comprising the following sequence SEQ ID No63 (positions 1024 to 1037 according to Desjardins and Morais, 1990, J. Mol. Biol., 212, 599-634):
CTC ACC GCA CAA ATA
or constituted by the following sequence SEQ ID No25 (positions 1019 to 1037 according to Desjardins and Morais, 1990, J. Mol. Biol., 212, 599-634):
ACA AAC TCA CCG CAC AAA TA
or those,
(4)—presenting a sequence identity of at least 80%, preferably 90% and advantageously 95% with an oligonucleotide constituted by a sequence of approximately 15 to 25 nucleotides, in particular 20 to 25 nucleotides, contained in the following sequence SEQ ID No64 (positions 1167 to 1192 according to Desjardins and Morais, 1990, J. Mol. Biol., 212, 599-634):
TGT AAR TAW CAA TAT AAA TAA TAT A
in which R is A or G, W is A or T,
or comprising the following sequence SEQ ID No65 (positions 1177 to 1192 according to
AAT ATA AAT AAT ATA
or constituted by the following sequence SEQ ID No26 (positions 1172 to 1191 according to Desjardins and Morais, 1990, J. Mol. Biol., 212, 599-634):
TTA ACA ATA TAA ATA ATA TA
or those,
(5)—presenting a sequence identity of at least 80%, preferably 90% and advantageously 95%, with an oligonucleotide constituted by a sequence of approximately 15 to 25 nucleotides, in particular 20 to 25 nucleotides, contained in the following sequence SEQ ID No70 (positions 684 to 709 according to Desjardins and Morais, 1990, J. Mol. Biol., 212, 599-634):
CCT TCM CAG TGM BKK YGS CRG RGT V
in which M is A or C, B is C, G or T, K is G or T, Y is C or T, S is C or G, R is A or G, V is A, C or G.
or comprising the following sequence SEQ ID No71 (positions 694 to 709 according to Desjardins and Morais, 1990, J. Mol. Biol., 212, 599-634):
GCC GTC GCC AGA GTA
or constituted by the following sequence SEQ ID No72 (positions 689 to 709 according to Desjardins and Morais, 1990, J. Mol. Biol., 212, 599-634):
ACA GTG CCG TCG CCA GAG TA
or those,
(6)—presenting a sequence identity of at least 80%, preferably 90% and advantageously 95%, with an oligonucleotide constituted by a sequence of approximately 15 to 25 nucleotides, in particular 20 to 25 nucleotides, contained in the following sequence SEQ ID No 73 (positions 898 to 923 according to Desjardins and Morais, 1990, J. Mol. Biol., 212, 599-634):
RRW WRT RGW KGR RGK GRR WWT TNR Y
in which R is A or G, W is A or T, K is G or T, N is A, C, G or T, Y is C or T,
or comprising the following sequence SEQ ID No74 (positions 898 to 913 according to Desjardins and Morais, 1990, J. Mol. Biol., 212, 599-634)
GAA GTG AAA ATT AGC
or constituted by the following sequence SEQ ID No75 (positions 898 to 918 according to Desjardins and Morais, 1990, J. Mol. Biol., 212, 599-634)
TAG AGG AAG TGA AAA TTA GC
or those,
(7)—presenting a sequence identity of at least 80%, preferably 90% and advantageously 95% with an oligonucleotide constituted by a sequence of approximately 15 to 25 nucleotides, in particular 20 to 25 nucleotides, contained in the following sequence SEQ ID No66 (positions 879 to 898 according to Desjardins and Morais, 1990, J. Mol. Biol., 212, 599-634):
TGA ATG CTT GYY GGA CAT WAA TAC C
in which Y is C or T, W is A or T,
or comprising the following sequence SEQ ID No67 (positions 888 to 898 according to Desjardins and Morais, 1990, J: Mol. Biol., 212, 599-634):
CTG GAC ATA AAT ACC
or constituted by the following sequence SEQ ID No27 (positions 884 to 898 according to Desjardins and Morais, 1990, J. Mol. Biol., 212, 599-634):
GCT TGC TGG ACA TAA ATA CC
or those,
(8)—presenting a sequence identity of at least 80%, preferably 90% and advantageously 95% with an oligonucleotide constituted by a sequence of approximately 15 to 25 nucleotides, in particular 20 to 25 nucleotides, contained in the following sequence SEQ ID No68 (positions 1077 to 1101 according to Desjardins and Morais, 1990, J. Mol. Biol., 212, 599-634):
CTA TAA TAK GRT GTG GGG CGT GTT G
in which K is G or T, R is A or G
or comprising the following sequence SEQ ID No69 (positions 1087 to 1101 according to Desjardins and Morais, 1990, J. Mol. Biol., 212, 599-634):
TTG TGG GGC GTG TTG
or constituted by the following sequence SEQ ID No28 (positions 1082 to 1101 according to Desjardins and Morais, 1990, J. Mol. Biol., 212, 599-634):
ATA AGT TGT GGG GCG TGT TG

10. Primer pairs characterized in that they are constituted:
by any one of the oligonucleotides SEQ ID No23, SEQ ID No58, SEQ ID No59, and any one of the oligonucleotides SEQ ID No24, SEQ ID No60, SEQ ID No61 according to claim 9 or,
by any one of the oligonucleotides SEQ ID No25, SEQ ID No62, SEQ ID No63, and any one of the oligonucleotides SEQ ID No26, SEQ ID No64, SEQ ID No65 according to claim 9 or,
by any one of the oligonucleotides SEQ ID No70, SEQ ID No71, SEQ ID No72, and any one of the oligonucleotides SEQ ID No73, SEQ ID No74, SEQ ID No75 as defined above or,
by any one of the oligonucleotides SEQ ID No27, SEQ ID No66, SEQ ID No67, and any one of the oligonucleotides SEQ ID No28, SEQ ID No68, SEQ ID No69 according to claim 9,
and advantageously constituted by the pair of oligonucleotides chosen from the following pairs:
SEQ ID No23 and SEQ ID No24,
SEQ ID No25 and SEQ ID No26,
SEQ ID No72 and SEQ ID No75,
SEQ ID No27 and SEQ ID No28.

11. Method according to claim 1, characterized in that the amplification of the said DNA sequence specific to the genome of birds, in particular of Anseriformes, Galliformes or Struthioniformes, is carried out by the polymerase chain (PCR) amplification method, comprising a repetition of the cycle of the following steps:
heating of the DNA extracted from the sample of organic material, so as to separate the DNA into two single-stranded strands,
hybridization of oligonucleotide primers according to claims 7 to 10 to the single-stranded DNA strands at an adequate temperature and,
elongation of the said oligonucleotide primers by a polymerase at an adequate temperature, in order to obtain an amplified DNA sequence or DNA fragment specific to the genome of birds, in particular of Anseriformes, Galliformes or Struthioniformes.

12. Method according to claim 1, characterized in that the amplified DNA fragment obtained is identified:
by sequencing of the said amplified fragment,
by gel electrophoresis of the fragments obtained after digestion using restriction enzymes of the said amplified fragment,
directly, by simple visualization of the presence of the said amplified fragment by gel electrophoresis.

13. Method according to claim 1, characterized in that the DNA extracted from the sample of organic material is:
non-degraded DNA derived in particular from a fresh sample or,
degraded DNA, derived in particular from a processed, in particular cooked, freeze-dried, dried, pickled, appertized or pasteurized, sample.

14. DNA fragment as amplified at the end of the method according to claim 1, characterized in that it comprises approximately 500 to approximately 100 base pairs.

15. DNA fragment according to claim 14, characterized in that it presents a sequence identity of at least 80%, preferably 90% and advantageously 95% with:
the following SEQ ID No6:

| | | | |
|---|---|---|---|
| ACYAGCTTCA | GGCCCATACG | TTCCCCCTAA | ACCCCTCGCC |
| CTCCTCACAT | TTTTGCGCCT | | |
| CTGGTTCCTC | GGTCAGGGCC | ATCMATTGGG | TTCACTCACC |
| YCYMYTYGCC | YTTCAAAGTG | | |
| GCATCTGTGG | ANKACBTYCA | CCWYYYCRRT | GCGTWATCGC |
| GGCATBYTYM | ASYWTTTWSM | | |
| CGCCTYTGGT | TCYMYTTHTY | TYT | |

in which Y is C or T, M is A or C, K is G or T, B is C, G or T, R is A or G, W is A or T, S is C or G, H is A, C or T, N is A, C, G or T,
the said sequence SEQ ID No6 comprising 203 base pairs,
or the following SEQ ID No7:

| | | | |
|---|---|---|---|
| ACYAGCTTCA | GGCCCATACG | TTCCCCCTAA | ACCCCTCGCC |
| CTCCTCACAT | TTTTGCGCCT | | |
| CTGGTTCCTC | GGTCAGGGCC | ATCMATTGGG | TTCACTCACC |
| YCYMYTYGCC | YTTCAAAGTG | | |
| GCATCTGTGG | ANKACBT | | |

in which Y is C or T, M is A or C, N is A, C, G or T, K is G or T, B is C, G or T,
the said sequence SEQ ID No7 comprising 137 base pairs,
or the following SEQ ID No8:

| | | | |
|---|---|---|---|
| AACCCCTCGC | CCTCCTCACA | TTTTTGCGCC | TCTGGTTCCT |
| CGGTCAGGGC | CATCMATTGG | | |
| GTTCACTCAC | CYCYMYTYGC | CYTTCAAAGT | GGCATCTGTG |
| GANKACBTYC | ACCWYYYCRR | | |
| TGCGTWATCG | CGGCATBYTY | MASYWTTTWS | MCGCCTYTGG |
| TTCYMYTTHT | YTYT | | |

in which Y is C or T, M is A or C, N is A, C, G or T, K is G or T, B is C, G or T, R is A or G, W is A or T, S is C or G, H is A, C or T,
the said sequence SEQ ID No8 comprising 174 base pairs,
or the following SEQ ID No9:

| | | | |
|---|---|---|---|
| AACCCCTCGC | CCTCCTCACA | TTTTTGCGCC | TCTGGTTCCT |
| CGGTCAGGGC | CATCMATTGG | | |
| GTTCACTCAC | CYCYMYTYGC | CYTTCAAAGT | GGCATCTGTG |
| GANKACBT | | | |

in which Y is C or T, M is A or C, N is A, C, G or T, K is G or T, B is C, G or T,
the said sequence SEQ ID No9 comprising 108 base pairs,
or the following SEQ ID No10:

| | | | |
|---|---|---|---|
| ATCMATTGGG | TTCACTCACC | YCYMYTYGCC | YTTCAAAGTG |
| GCATCTGTGG | ANKACBTYCA | | |
| CCWYYYCRRT | GCGTWATCGC | GGCATBYTYM | ASYWTTTWSM |
| CGCCTYTGGT | TCYMYTTHTY | | |
| TYT | | | |

in which Y is C or T, M is A or C, N is A, C, G or T, K is G or T, B is C, G or T, R is A or G, W is A or T, S is C or G, H is A, C or T,
the said sequence SEQ ID No110 comprising 123 base pairs,
or the following SEQ ID No13:

| | | | |
|---|---|---|---|
| YMYYKRYACT | GATGCACTTK | GDYCDCATTY | RGTTRWKGYY |
| MYNTCCRCHV | HYCYABNBWH | | |
| WVRTGYTRYY | NRGTGAATGC | TYGHHGGACA | TADYWYWAWM |
| MAWHWWMHYW | YYHWSYRYWW | | |
| YYYHYRSRMH | HHMNRAAYDM | AACCARHRDW | YTTYMDCHRW |
| THWDHWWHMM | WHYWYVTWDY | | |
| RTMWWHYHHV | HMHHHHMHWM | HHBHAWWHHH | HNHNHYNHWA |
| YYBCHNNYHH | HWAWYNYSYY | | |
| WHHYNWBVHW | HHNBYBDMDM | DWCHDWMMNR | WWHDMAYDDH |
| DYVDHHYAWM | HTTATTAKAG | | |
| RAACWCCAGY | ACYARVVVHD | VWHNHAMHYN | RRHRWHHMYY |
| AYWAHYYHVH | YNTYNMNAMH | | |
| HNBNVYHYRD | HYHRYYARYB | RMHYVHHHYR | YYCDCMTWSY |
| WYMMHNHH | | | |

in which Y is C or T, M is A or C, N is A, C, G or T, K is G or T, B is C, G or T, R is A or G, W is A or T, S is C or G, D is A, G or T, V is A, C or G, H is A, C or T,
the said sequence SEQ ID No13 comprising 408 base pairs,
or fragments resulting from one or more deletions of contiguous or non-contiguous nucleotides of the said fragment of 408 base pairs comprising or constituted by SEQ ID No13, and capable of being obtained by amplification of mtDNA derived from birds, using the oligonucleotides SEQ ID No11 and SEQ ID No12 as defined in claim 7, and in particular fragments of:
331 base pairs (SEQ ID NO: 77):

| | | | |
|---|---|---|---|
| YMYYKRCTGA | TGCACTTKGD | YCDCATTYRG | TTRWKGYYMY |
| CCRCHVHYCY | ABNBWHWVRT | | |
| GYTRYYNRGT | GAATGCTYGH | HGGACATADY | WYWAWMMAWH |
| WMHYWYYHWS | YRYWWYYYHY | | |
| RSRMHHHMRA | AYDMAACCAR | HRDWYTTYMD | CHRWTHDYRT |
| MWWHYHVHMH | HNHWAYYBCH | | |
| NNYHDMDWCH | DWMMRWWHDM | AYDDHDYVDH | HYAWHTTATT |
| AKAGRAACWC | CAGYACYARV | | |
| VVHDVWHNHA | MHYNRRHRWH | MYYAYWAHYY | HVHYNTYNMN |
| AMHHNBNVYH | YRDHYHRYYA | | |
| RYBRMHYVHH | HYRYYCDCMT | WSYWYMMHNH | H |

in which Y is C or T, M is A or C, N is A, C, G or T, K is G or T, B is C, G or T, R is A or G, W is A or T, S is C or G, D is A, G or T, V is A, C or G, H is A, C or T,
328 base pairs (SEQ ID NO: 78):

| | | | |
|---|---|---|---|
| YMYYKRYACT | GATGCACTTK | GDYCDCATTY | RGTTRWKGYY |
| MYNTCCRCHV | HYCYABNBWH | | |
| WVRTGYTRYY | NRGTGAATGC | TYGHHGGACA | TAMHHHMNRA |
| AYDMAACCAY | DMAACHRHYH | | |
| HVHMHHHNHN | HYNHHNNYRH | HWAYYBCHNN | YHHWAWYMYS |
| YYWHHYNWBV | HWHNBYBDMD | | |
| MDWCHDWMNR | WWHDMAYDDH | DYVDHHYAWM | HTTATTAKAG |
| RAACWCCAGY | ACYARVVVHD | | |
| VWHNHAMHYN | RRHRWHHMYY | AYWAHYYHVH | YNTYNMNAMH |
| HNBNVYHYRD | HYHRYYARYB | | |
| RMHYVHHHYR | YYCDCMTWSY | WYMMHNHH | |

in which Y is C or T, M is A or C, N is A, C, G or T, K is G or T, B is C, G or T, R is A or G, W is A or T, S is C or G, D is A, G or T, V is A, C or G, H is A, C or T,
or 288 base pairs (SEQ ID NO: 79):

| | | | |
|---|---|---|---|
| YMYYKRYACT | GATGCACTTK | GDYCDCATTY | RGTTRWKGYY |
| MYNTCCRCHV | HYCYABNBWH | | |
| WVRTGYTRYY | NRGTGAATGC | TYGHHGGACA | TAMHHHMNRA |
| AYDMAACHRH | YHHVHMHHHN | | |
| HNHYNHHNNY | HHHWAYWBVH | WHWCHDWMMN | RWWHDMAYDD |
| HDYVDHHYAW | MHTTATTAKA | | |
| GRAACWCCAG | YACYARVVVH | DVWHNHAMHY | NRRHRWHHMY |
| YAYWAHYYHV | HYNTYNMNMH | | |
| HNBNVYHYRD | HYHRYYARYB | RMHYVHHHYR | YYCDCMTWSY |
| WYMMHNHH | | | |

in which Y is C or T, M is A or C, N is A, C, G or T, K is G or T, B is C, G or T, R is A or G, W is A or T, S is C or G, D is A, G or T, V is A, C or G, H is A, C or T,
or the following SEQ ID No20:

| | | | |
|---|---|---|---|
| HWBDNHMRMG | RNCATHHHHN | NYHVHBDNAH | NNNHCHHCCA |
| VVRMHHHNNV | RTGMAYGHYC | | |
| YHRNDVHHHH | NHVNHVVMHV | VMHVNVHNHN | NHHHNHVHVV |
| RWNMACMARR | RCMMHDHNNR | | |
| VTGAATGCTH | NMHRRACAWR | DMNYNDVHAH | HMHTCYDDNB |
| YYBHHTCCAC | CCRMMCATWH | | |

in which Y is C or T, M is A or C, N is A, C, G or T, B is C, G or T, R is A or G, W is A or T, D is A, G or T, V is A, C or G, H is A, C or T,
the said sequence SEQ ID No20 comprising 180 base pairs,
or the following SEQ ID No21:

| | | | |
|---|---|---|---|
| WKGYYMYNTC | CRCHVHYCYA | BNBWHWVRTG | YTRYYNRGTG |
| AATGCTYGHH | GGACATAMHH | | |
| HMNRAAYDMA | ACHRHYHHVH | MHHHNHNHNH | HNNYHHHWAY |
| WBVHWHWCHD | WMMNRWWHDM | | |
| AYDDHDYVDH | HYAWMHTTAT | TAKAGRAACW | CCAGYACYAR |
| VVVHDVWHNH | AMHYNRRHRW | | |
| HHMYYAYWAH | Y | | |

in which Y is C or T, M is A or C, N is A, C, G or T, B is C, G or T, R is A or G, W is A or T, D is A, G or T, V is A, C or G, H is A, C or T, K is G or T,
the said sequence SEQ ID No21 comprising 191 base pairs,
or the following SEQ ID No22:

| | | | |
|---|---|---|---|
| RTGAAYGHYC | YHRNDVHHNH | MVHNHNVMHV | VNHNHHNHNH |
| VVVSWNMARD | SCMNHNNTTM | | |
| VTGAATGCTH | NMHRRACAWD | MNYNDVHAHH | MHTCYNDNBH |
| YBHHTCCACC | CRMMCATWHM | | |
| TYATGRAVYY | NCGTAYCARR | TGGATTTATT | RRYCGTACWC |
| CTCACGTGAA | ATCAGC | | |

in which Y is C or T, M is A or C, N is A, C, G or T, B is C, G or T, R is A or G, W is A or T, D is A, G or T, V is A, C or G, H is A, C or T, S is C or G,
the said sequence SEQ ID No22 comprising 176 base pairs.

16. DNA fragment according to claim 14, characterized in that it presents a sequence identity of at least 80%, preferably 90% and advantageously 95% with:
the following SEQ ID No29:
KMAWVNGGHM WTVACHYATA TTCCMCWTYY CKYMCHAHVB
MCHYTVDATG MAYSMTSHHRK RCMHVCTMHN TTHCVYDHMC
CATAGABAGT TCCWAACHAC WATCRRKVMM CMTAACTATG
AATGGTTRCVG HACATAAATC TCACTCTCAT
GTTCTCCCCC CAACAAGTC CCTAACTATG AATGGTTACAG
GACATACATT TDACTAYCAT GTTCTAACCS
ATTTGGTT
in which Y is C or T, M is A or C, N is A, C, G or T, B is C, G or T, R is A or G, W is A or T, D is A, G or T, V is A, C or G, H is A, C or T, S is C or G, K is G or T
the said sequence SEQ ID No29 comprising 231 base pairs,
or the following SEQ ID No30:
TACAWACYMW CGYAWWRRWD CCCTYAMAYY AYAHAAMCGY
YTATCGYWTA DTATATATAC ATTRYWRYDY AYYMYAYMAT
TATTAKAGAA ACYCCMCTAC CRAAACMAWC AWHARARRCW
WAHADHWAHA TKCMRCDYAW VBYHCYTCAS AWRCMWWBRT
KRTWTATATT GTTAA
in which Y is C or T, M is A or C, B is C, G or T, R is A or G, W is A or T, D is A, G or T, V is A, C or G, H is A, C or T, S is C or G, K is G or T,
the said sequence SEQ ID No30 comprising 175 base pairs,
or the following SEQ ID No76:
MCAGTGMSKK YGSCRGRGTV CYAYTCAAKY KRAGCCTCGA
YTACWCCTGC GTTRCGYSCT ATYCTAKWYY TCHVGKRTYM
CTCRATGAKA CGGTTKGCGT RTATKKGGWA TCAYYTTGAC
ACTGATGCAC TTTGGAYCRC ATTYRGTTAW KGYTCTYCCR
CMSYYCYNKW WARTRGKGYT ATWTAGTGAA TGCTYGHYGG
ACATAYYTTA WSYNAAWWYY CMCYYCMWCY A
in which M is A or C, S is C or G, K is G or T, Y is C or T, R is A or G, V is A, C or G, W is A or T, N is A, C, T or G,
the said sequence SEQ ID No76 comprising 231 base pairs,
or the following SEQ ID No31:
TGAATGCTTG YYGGACATWW TWYMAYYWAT TWTCACTTCC
WCTGAYTTTY YTMACAMMAC YAGGAYWMAW YTYYTWYDTY
DKTWYWAAYW YWTKWTTTVW TYAYWTYAAW MACATYTTTT
AMYTAWAYTA MCYWTACAWA CYMWCMGYAY WRRWTDTYAM
AYYAAHAAMC GYYCGYWTAD TATATATACA TARYWRYTGY
RYYTMYAYCM ATTATT
in which Y is C or T, M is A or C, R is A or G, W is A or T, D is A, G or T, V is A, C or G, H is A, C or T,
the said sequence SEQ ID No31 comprising 216 base pairs.

17-18. (canceled)


SEQUENCE LISTING (96 sequences)

SEQ ID NO: 1 - DNA, 20 bp, Artificial
  Description of Artificial Sequence Synthetic oligonucleotide
actagcttcaggcccatacg

SEQ ID NO: 2 - DNA, 20 bp, Artificial
  Description of Artificial Sequence Synthetic oligonucleotide
aacccctcgccctcctcaca

SEQ ID NO: 3 - DNA, 20 bp, Artificial
  Description of Artificial Sequence Synthetic oligonucleotide
atcaattgggttcactcacc

SEQ ID NO: 4 - DNA, 20 bp, Artificial
  Description of Artificial Sequence Synthetic oligonucleotide
aagtattccacagatgccac

SEQ ID NO: 5 - DNA, 20 bp, Artificial
  Description of Artificial Sequence Synthetic oligonucleotide
aaaaataaaaggaaccagag

SEQ ID NO: 6 - DNA, 203 bp, Artificial
  Description of Artificial Sequence DNA fragment amplified from the genome of birds
acyagcttcaggcccatacgttccccctaaacccctcgccctcctcacatttttgcgcctctggttcctcggtcagggccatcmattgggttcactcaccycymytygccyttcaaagtggcatctgtggankacbtycaccwyyycrrtgcgtwatcgcggcatbytymasywtttwsmcgcctytggttcymytthtytyt

SEQ ID NO: 7 - DNA, 137 bp, Artificial
  Description of Artificial Sequence DNA fragment amplified from the genome of birds
acyagcttcaggcccatacgttccccctaaacccctcgccctcctcacatttttgcgcctctggttcctcggtcagggccatcmattgggttcactcaccycymytygccyttcaaagtggcatctgtggankacbt

SEQ ID NO: 8 - DNA, 174 bp, Artificial
  Description of Artificial Sequence DNA fragment amplified from the genome of birds
aacccctcgccctcctcacatttttgcgcctctggttcctcggtcagggccatcmattgggttcactcaccycymytygccyttcaaagtggcatctgtggankacbtycaccwyyycrrtgcgtwatcgcggcatbytymasywtttwsmcgcctytggttcymytthtytyt

SEQ ID NO: 9 - DNA, 108 bp, Artificial
  Description of Artificial Sequence DNA fragment amplified from the genome of birds
aacccctcgccctcctcacatttttgcgcctctggttcctcggtcagggccatcmattgggttcactcaccycymytygccyttcaaagtggcatctgtggankacbt

SEQ ID NO: 10 - DNA, 123 bp, Artificial
  Description of Artificial Sequence DNA fragment amplified from the genome of birds
atcmattgggttcactcaccycymytygccyttcaaagtggcatctgtggankacbtycaccwyyycrrtgcgtwatcgcggcatbytymasywtttwsmcgcctytggttcymytthtytyt

SEQ ID NO: 11 - DNA, 20 bp, Artificial
  Description of Artificial Sequence Synthetic oligonucleotide
ccttgacactgatgcacttt

SEQ ID NO: 12 - DNA, 20 bp, Artificial
  Description of Artificial Sequence Synthetic oligonucleotide
tgtggtaagctatgtggacg

SEQ ID NO: 13 - DNA, 408 bp, Artificial
  Description of Artificial Sequence DNA fragment amplified from the genome of birds
ymyykryactgatgcacttkgdycdcattyrgttrwkgyymyntccrchvhycyabnbwhwvrtgytryynrgtgaatgctyghhggacatadywywawmmawhwwmhywyyhwsyrywwyyyhyrsrmhhhmnraaydmaaccarhrdwyttymdchrwthwdhwwhmmwhywyvtwdyrtmwwhyhhvhmhhhhmhwmhhbhawwhhhhnhnhynhwayybchnnyhhhwawynysyywhhynwbvhwhhnbybdmdmdwchdwmmnrwwhdmayddhdyvdhhyawmhttattakagraacwccagyacyarvvvhdvwhnhamhynrrhrwhhmyyaywahyyhvhyntynmnamhhnbnvyhyrdhyhryyarybrmhyvhhhyryycdcmtwsywymmhnhh

SEQ ID NO: 14 - DNA, 21 bp, Artificial
  Description of Artificial Sequence Synthetic oligonucleotide
catgtcaacggacatacccta

SEQ ID NO: 15 - DNA, 19 bp, Artificial
  Description of Artificial Sequence Synthetic oligonucleotide
taatgggtgggtggagagg

SEQ ID NO: 16 - DNA, 21 bp, Artificial
  Description of Artificial Sequence Synthetic oligonucleotide
atgctctcctccgcagccctt

SEQ ID NO: 17 - DNA, 20 bp, Artificial
  Description of Artificial Sequence Synthetic oligonucleotide
gattgtcgttagattacgct

SEQ ID NO: 18 - DNA, 19 bp, Artificial
  Description of Artificial Sequence Synthetic oligonucleotide
atgaatgttctaggaccat

SEQ ID NO: 19 - DNA, 20 bp, Artificial
  Description of Artificial Sequence Synthetic oligonucleotide
tctgatttcacgtgaggagt

SEQ ID NO: 20 - DNA, 180 bp, Artificial
  Description of Artificial Sequence DNA fragment amplified from the genome of birds
hwbdnhmrmgrncathhhhnnyhvhbdnahnnnhchhccavvrmhhhnnvrtgmayghycyhrndvhhhhnhvnhvvmhvvmhvnvhnhnnhhhnhvhvvrwnmacmarrrcmmhdhnnrvtgaatgcthnmhrracawrdmnyndvhahhmhtcyddnbyybhhtccacccrmmcatwh

SEQ ID NO: 21 - DNA, 191 bp, Artificial
  Description of Artificial Sequence DNA fragment amplified from the genome of birds
wkgyymyntccrchvhycyabnbwhwvrtgytryynrgtgaatgctyghhggacatamhhhmnraaydmaachrhyhhvhmhhhnhnhnhhnnyhhhwaywbvhwhwchdwmmnrwwhdmayddhdyvdhhyawmhttattakagraacwccagyacyarvvvhdvwhnhamhynrrhrwhhmyyaywahy

SEQ ID NO: 22 - DNA, 176 bp, Artificial
  Description of Artificial Sequence DNA fragment amplified from the genome of birds
rtgaayghycyhrndvhhnhmvhnhnvmhvvnhnhhnhnhvvvswnmardscmnhnnttmvtgaatgcthnmhrracawdmnyndvhahhmhtcyndnbhybhhtccacccrmmcatwhmtyatgravyyncgtaycarrtggatttattrrycgtacwcctcacgtgaaatcagc

SEQ ID NO: 23 - DNA, 20 bp, Artificial
  Description of Artificial Sequence Synthetic oligonucleotide
atacgggcattaacctatat

SEQ ID NO: 24 - DNA, 20 bp, Artificial
  Description of Artificial Sequence Synthetic oligonucleotide
cataaccaaatgggttacat

SEQ ID NO: 25 - DNA, 20 bp, Artificial
  Description of Artificial Sequence Synthetic oligonucleotide
acaaactcaccgcacaaata

SEQ ID NO: 26 - DNA, 20 bp, Artificial
  Description of Artificial Sequence Synthetic oligonucleotide
ttaacaatataaataatata

SEQ ID NO: 27 - DNA, 20 bp, Artificial
  Description of Artificial Sequence Synthetic oligonucleotide
gcttgctggacataaatacc

SEQ ID NO: 28 - DNA, 20 bp, Artificial
  Description of Artificial Sequence Synthetic oligonucleotide
ataagttgtggggcgtgttg

SEQ ID NO: 29 - DNA, 231 bp, Artificial
  Description of Artificial Sequence DNA fragment amplified from the genome of birds
kmawvngghmwtvachyatattccmcwtyyckymchahvbmchytvdatgmaysmtshhrkrcmhvctmhntthcvydhmccatagabagttccwaachacwatcrrkvmmcmtaactatgaatggttrcvghacataaatctcactctcatgttctccccccaacaagtcacctaactatgaatggttacaggacatacatttdactaycatgttctaaccsatttggtt

SEQ ID NO: 30 - DNA, 175 bp, Artificial
  Description of Artificial Sequence DNA fragment amplified from the genome of birds
tacawacymwcgyawwrrwdccctyamayyayahaamcgyytatcgywtadtatatatacattrywrydyayymyaymattattakagaaacyccmctaccraaacmawcawhararrcwwahadhwahatkcmrcdyawvbyhcytcasawrcmwwbrtkrtwtatattgttaa

SEQ ID NO: 31 - DNA, 216 bp, Artificial
  Description of Artificial Sequence DNA fragment amplified from the genome of birds
tgaatgcttgyyggacatwwtwymayywattwtcacttccwctgaytttyytmacammacyaggaywmawytyytwydtydktwywaaywywtkwtttvwtyaywtyaawmacatyttttamytawaytamcywtacawacymwcmgyaywrrwtdtyamayyaahaamcgyycgywtadtatatatacatarywrytgyryytmyaycmattatt

SEQ ID NO: 32 - DNA, 25 bp, Artificial
  Description of Artificial Sequence Synthetic oligonucleotide
acgtgacyagcytcaggcccatacg

SEQ ID NO: 33 - DNA, 15 bp, Artificial
  Description of Artificial Sequence Synthetic oligonucleotide
cttcaggcccatacg

SEQ ID NO: 34 - DNA, 25 bp, Artificial
  Description of Artificial Sequence Synthetic oligonucleotide
ccctaaacccctcgccctcctcaca

SEQ ID NO: 35 - DNA, 15 bp, Artificial
  Description of Artificial Sequence Synthetic oligonucleotide
ctcgccctcctcaca

SEQ ID NO: 36 - DNA, 25 bp, Artificial
  Description of Artificial Sequence Synthetic oligonucleotide
gggccatcmattgggttcactcacc

SEQ ID NO: 37 - DNA, 15 bp, Artificial
  Description of Artificial Sequence Synthetic oligonucleotide
ttgggttcactcacc

SEQ ID NO: 38 - DNA, 25 bp, Artificial
  Description of Artificial Sequence Synthetic oligonucleotide
ggtrravstmntccacagatgccac

SEQ ID NO: 39 - DNA, 15 bp, Artificial
  Description of Artificial Sequence Synthetic oligonucleotide
ttccacagatgccac

SEQ ID NO: 40 - DNA, 25 bp, Artificial
  Description of Artificial Sequence Synthetic oligonucleotide
cmggaararadaarwrgaaccagag

SEQ ID NO: 41 - DNA, 16 bp, Artificial
  Description of Artificial Sequence Synthetic oligonucleotide
ataaaaagaaccagag

SEQ ID NO: 42 - DNA, 25 bp, Artificial
  Description of Artificial Sequence Synthetic oligonucleotide
wvkcaymyykryactgatgcacttt

SEQ ID NO: 43 - DNA, 15 bp, Artificial
  Description of Artificial Sequence Synthetic oligonucleotide
acactgatgcacttt

SEQ ID NO: 44 - DNA, 25 bp, Artificial
  Description of Artificial Sequence Synthetic oligonucleotide
gcyttddndkkrwrswatgtggacg

SEQ ID NO: 45 - DNA, 15 bp, Artificial
  Description of Artificial Sequence Synthetic oligonucleotide
taagctatgtggacg

SEQ ID NO: 46 - DNA, 25 bp, Artificial
  Description of Artificial Sequence Synthetic oligonucleotide
mtnmhmbdnhmvhvnncatacccta

SEQ ID NO: 47 - DNA, 15 bp, Artificial
  Description of Artificial Sequence Synthetic oligonucleotide
aacggacatacccta

SEQ ID NO: 48 - DNA, 25 bp, Artificial
  Description of Artificial Sequence Synthetic oligonucleotide
catragnwawkgkygggtggagagg

SEQ ID NO: 49 - DNA, 15 bp, Artificial
  Description of Artificial Sequence Synthetic oligonucleotide
gggtgggtggagagg

SEQ ID NO: 50 - DNA, 25 bp, Artificial
  Description of Artificial Sequence Synthetic oligonucleotide
gttawkgyymynctccgcrgccctt

SEQ ID NO: 51 - DNA, 15 bp, Artificial
  Description of Artificial Sequence Synthetic oligonucleotide
tcctccgcagccctt

SEQ ID NO: 52 - DNA, 25 bp, Artificial
  Description of Artificial Sequence Synthetic oligonucleotide
dtwatgatthtyrttadattacgct

SEQ ID NO: 53 - DNA, 15 bp, Artificial
  Description of Artificial Sequence Synthetic oligonucleotide
tcgttagattacgct

SEQ ID NO: 54 - DNA, 24 bp, Artificial
  Description of Artificial Sequence Synthetic oligonucleotide
aayghatgaayghycagndaccat

SEQ ID NO: 55 - DNA, 15 bp, Artificial
  Description of Artificial Sequence Synthetic oligonucleotide
atgttctaggaccat

SEQ ID NO: 56 - DNA, 25 bp, Artificial
  Description of Artificial Sequence Synthetic oligonucleotide
cgggttctgatttcacgtgaggagt

SEQ ID NO: 57 - DNA, 15 bp, Artificial
  Description of Artificial Sequence Synthetic oligonucleotide
tttcacgtgaggagt

SEQ ID NO: 58 - DNA, 25 bp, Artificial
  Description of Artificial Sequence Synthetic oligonucleotide
atatgkmvncggcattaacctatat

SEQ ID NO: 59 - DNA, 15 bp, Artificial
  Description of Artificial Sequence Synthetic oligonucleotide
ggcattaacctatat

SEQ ID NO: 60 - DNA, 25 bp, Artificial
  Description of Artificial Sequence Synthetic oligonucleotide
gcgagyataaccaaatsggttacat

SEQ ID NO: 61 - DNA, 15 bp, Artificial
  Description of Artificial Sequence Synthetic oligonucleotide
ccaaatgggttacat

SEQ ID NO: 62 - DNA, 25 bp, Artificial
  Description of Artificial Sequence Synthetic oligonucleotide
tacatacawacymwccgcacaaata

SEQ ID NO: 63 - DNA, 15 bp, Artificial
  Description of Artificial Sequence Synthetic oligonucleotide
ctcaccgcacaaata

SEQ ID NO: 64 - DNA, 25 bp, Artificial
  Description of Artificial Sequence Synthetic oligonucleotide
tgtaartawcaatataaataatata

SEQ ID NO: 65 - DNA, 15 bp, Artificial
  Description of Artificial Sequence Synthetic oligonucleotide
aatataaataatata

SEQ ID NO: 66 - DNA, 25 bp, Artificial
  Description of Artificial Sequence Synthetic oligonucleotide
tgaatgcttgyyggacatwaatacc

SEQ ID NO: 67 - DNA, 15 bp, Artificial
  Description of Artificial Sequence Synthetic oligonucleotide
ctggacataaatacc

SEQ ID NO: 68 - DNA, 25 bp, Artificial
  Description of Artificial Sequence Synthetic oligonucleotide
ctataatakgrtgtggggcgtgttg

SEQ ID NO: 69 - DNA, 15 bp, Artificial
  Description of Artificial Sequence Synthetic oligonucleotide
ttgtggggcgtgttg

SEQ ID NO: 70 - DNA, 25 bp, Artificial
  Description of Artificial Sequence Synthetic oligonucleotide
ccttcmcagtgmbkkygscrgrgtv

SEQ ID NO: 71 - DNA, 15 bp, Artificial
  Description of Artificial Sequence Synthetic oligonucleotide
gccgtcgccagagta

SEQ ID NO: 72 - DNA, 20 bp, Artificial
  Description of Artificial Sequence Synthetic oligonucleotide
acagtgccgtcgccagagta

SEQ ID NO: 73 - DNA, 25 bp, Artificial
  Description of Artificial Sequence Synthetic oligonucleotide
rrwwrtrgwkgrrgkgrrwwttnry

SEQ ID NO: 74 - DNA, 15 bp, Artificial
  Description of Artificial Sequence Synthetic oligonucleotide
gaagtgaaaattagc

SEQ ID NO: 75 - DNA, 20 bp, Artificial
  Description of Artificial Sequence Synthetic oligonucleotide
tagaggaagtgaaaattagc

SEQ ID NO: 76 - DNA, 231 bp, Artificial
  Description of Artificial Sequence DNA fragment amplified from the genome of birds
mcagtgmskkygscrgrgtvcyaytcaakykragcctggaytacwcctgcgttrcgysctatyctakwyytchvgkrtymctcratgakacggttkgcgtrtatkkggwatcayyttgacactgatgcactttggaycrcattyrgttawkgytctyccrcmsyycynkwwartrgkgytatwtagtgaatgctyghyggacatayyttawsynaawwyycmcyycmwcya

SEQ ID NO: 77 - DNA, 331 bp, Artificial
  Description of Artificial Sequence DNA fragment amplified from the genome of birds
ymyykrctgatgcacttkgdycdcattyrgttrwkgyymyccrchvhycyabnbwhwvrtgytryynrgtgaatgctyghhggacatadywywawmmawhwmhywyyhwsyrywwyyyhyrsrmhhhmraaydmaaccarhrdwyttymdchrwthdyrtmwwhyhvhmhhnhwayybchnnyhdmdwchdwmmrwwhdmayddhdyvdhhyawhttattakagraacwccagyacyarvvvhdvwhnhamhynrrhrwhmyyaywahyyhvhyntynmnamhhnbnvyhyrdhyhryyarybrmhyvhhhyryycdcmtwsywymmhnhh

SEQ ID NO: 78 - DNA, 328 bp, Artificial
  Description of Artificial Sequence DNA fragment amplified from the genome of birds
ymyykryactgatgcacttkgdycdcattyrgttrwkgyymyntccrchvhycyabnbwhwvrtgytryynrgtgaatgctyghhggacatamhhhmnraaydmaaccaydmaachrhyhhvhmhhhnhnhynhhnnyrhhwayybchnnyhhwawymysyywhhynwbvhwhnbybdmdmdwchdwmnrwwhdmayddhdyvdhhyawmhttattakagraacwccagyacyarvvvhdvwhnhamhynrrhrwhhmyyaywahyyhvhyntynmnamhhnbnvyhyrdhyhryyarybrmhyvhhhyryycdcmtwsywymmhnhh

SEQ ID NO: 79 - DNA, 288 bp, Artificial
  Description of Artificial Sequence DNA fragment amplified from the genome of birds
ymyykryactgatgcacttkgdycdcattyrgttrwkgyymyntccrchvhycyabnbwhwvrtgytryynrgtgaatgctyghhggacatamhhhmnraaydmaachrhyhhvhmhhhnhnhynhhnnyhhhwaywbvhwhwchdwmmnrwwhdmayddhdyvdhhyawmhttattakagraacwccagyacyarvvvhdvwhnhamhynrrhrwhhmyyaywahyyhvhyntynmnmhhnbnvyhyrdhyhryyarybrmhyvhhhyryycdcmtwsywymmhnhh

SEQ ID NO: 80 - DNA, 103 bp, Anas platyrhynchos
aacggacccagagtgaatgctctaatacccaacacctcaacaccacataacatgcccccaaccagaacaaggccccataatgatgaatgcttgacagacatac

SEQ ID NO: 81 - DNA, 102 bp, Anas acuta
aacggacccaggatgaatgccccaacacccaacatccaacaccacatccacactctctcgaccagaacaaggccccataagatgaatgcttgacagacatac

SEQ ID NO: 82 - DNA, 102 bp, Cairina moschata
aaccactcaaggtgaacgctcccagacagcaacctccaaccaacagagcacagctccagaccaaaacaaggcccacgcaggatgaatgctcgatagacatac

SEQ ID NO: 83 - DNA, 104 bp, Aythya fuligula
aggtgactaaagatgaatgctcccaggcccactccagccaacagcaaccacacagcttcagaccataacaaggccccatacaagtgaatgctcgacagacatac

SEQ ID NO: 84 - DNA, 106 bp, Aix galericulata
aaccaactactagtgaatgctctaaggaccattccattacagctcacaatatcctagctcccgaccagaacaaggcccacgttgaatgaatgcttgctggacatac

SEQ ID NO: 85 - DNA, 105 bp, Calonetta leucophrys
acggaccctaaagtgaatgctcaagtactatcaacccacgccaggacagcccaaagctcaaaacctaaacaagaccacagcacagtgaatgctcgacagacataa

SEQ ID NO: 86 - DNA, 105 bp, Mergus cucullatus
aacaacattacagtgcatgtcctagaagacttaacctgtaccacgacaactaccctccagccaccaaaacaaggcccactacaagtgaatgctctacagacataa

SEQ ID NO: 87 - DNA, 101 bp, Melanitta nigra
aacaacttcacagtgaatgccctaaaagactaagccataccacaacagcccctgcacaccaccagaacaagaccccataacatgaatgctaacagacataa

SEQ ID NO: 88 - DNA, 102 bp, Branta leucopsis
aaccactacagcatgaatgctcctaaacaacccaacaagccccaaataacctcgcacgtacacataacaagaccccatacaatgaatgcttccaggacataa

SEQ ID NO: 89 - DNA, 105 bp, Anser anser
aaccacctaacatgaatgctctaggaccacaccccacaacacccaacacaactctagctcaagcacacaacaaggccccattttaatgaatgcttacaggacata

SEQ ID NO: 90 - DNA, 103 bp, Anser indicus
aaccacccggcatgaatgttctaggaccataccccacaacacccaatacaattccattcaagcgcataacaagaccccattttatgaatgctcacaggacatg

SEQ ID NO: 91 - DNA, 54 bp, Cygnus sp.
accacccccaaccacccacaagaccaccaaccaatgaatgctaacaagacatac

SEQ ID NO: 92 - DNA, 101 bp, Dendrocygna bicolor
aaccccaagacatgaatgatctaggactaaccccctaactcccatataaacagccaggcagacacccaaggcccacaacaatgaatgcttacaggacaata

SEQ ID NO: 93 - DNA, 119 bp, Gallus gallus
atagacagttccaaaccactatcaagccacctaactatgaatggttacaggacataaatctcactctcatgttctccccccaacaagtcacctaactatgaatggttacaggacataca

SEQ ID NO: 94 - DNA, 72 bp, Coturnix coturnix
accatacgttcacctagtaatagactttccactaacaggacaccataactatgaatggttgcaggacataag

SEQ ID NO: 95 - DNA, 104 bp, Struthio camelus
aaacagtaatagcttcaatacccgtactaaatactaccctgatatgccggacatgttcatggtaaatcctcagtaattgtacgataaactctcatttaatgtac

SEQ ID NO: 96 - DNA, 203 bp, Aix galericulata
actagcttcaggcccatacgttccccctaaacccctcgccctcctcacatttttgcgcctctggttcctcggtcagggccatcaattgggttcactcacctcctccttgcctttcaaagtggcatctgtggaagacctccaccatctcaatgcgtaatcgcggcatcctccagctttttggcgcctctggttcctcttatttt